(19) United States
(12) Patent Application Publication
(10) Pub. No.: US 2005/0009158 A1
(43) Pub. Date: Jan. 13, 2005

(54) Process for enzymatically resolving an enantiomeric mixture of alpha-hydroxy acids

(76) Inventors: Arindam Roy, Florence, SC (US); Shubhender Kapila, Rolla, MO (US); Paul K.S. Nam, Rolla, MO (US); Virgil J. Flanigan, Rolla, MO (US); Stephen J. Lorbert, St. Louis, MO (US); Charles S. Schasteen, St. Louis, MO (US)

Correspondence Address: SENNIGER POWERS LEAVITT AND ROEDEL, ONE METROPOLITAN SQUARE, 16TH FLOOR, ST LOUIS, MO, 63102, US

(21) Appl. No.: 10/795,790

(22) Filed: Mar. 8, 2004

Related U.S. Application Data
(60) Provisional application No. 60/452,959, filed on Mar. 7, 2003.
(60) Provisional application No. 60/453,355, filed on Mar. 10, 2003.

(51) Int. Cl. C12P 7/42

(52) U.S. Cl. 435/146; 562/401

(57) ABSTRACT

The present invention relates to a process for resolving an enantiomeric mixture of a-hydroxy acids or derivatives thereof through esterification and subsequent enzymatic hydrolysis of the α-hydroxy acids or derivatives. The present invention also relates to purified α-hydroxy acids or derivatives and methods of use thereof.

This application claims priority of U.S. patent application Ser. No. 60/452,959, filed Mar. 7, 2003 and U.S. patent application Ser. No. 60/453,355, filed Mar. 10, 2003.

### BACKGROUND OF THE INVENTION

The present invention relates to a process for resolving an enantiomeric mixture of α-hydroxy acids or derivatives thereof through esterification and subsequent enzymatic hydrolysis of the α-hydroxy acid esters or derivatives. The present invention also relates to purified α-hydroxy acids or derivatives and methods of use thereof.

It is known that the hydroxy analogs of certain α-amino acids, such as 2-hydroxy-4-methylthiobutanoic acid, the hydroxy analog of methionine, have biological activity comparable to or equivalent to the amino acids themselves. α-Amino acids and their hydroxy analogs have wide application in animal feed supplements, human nutrition, and as intermediates in the preparation of pharmaceutical peptides and other nutritional or pharmaceutical compositions. It is also known that, in the case of many amino acids, only one enantiomer, typically the L-form of the acid, exhibits biological activity. The same is generally the case with regard to the hydroxy analogs of such acids.

When animals metabolize feed sources that contain protein and α-amino acid nutrients, only the L-isomers of α-amino acids are utilized by the animal for incorporation into proteins. In an effort to improve nutrition, the diets of animals may be supplemented with α-hydroxy acids, more specifically, α-hydroxy analogs of naturally occurring α-amino acids. The manufacturing processes that synthesize these supplements typically create a racemic mixture of D- and L-isomers of α-hydroxy analogs of naturally occurring α-amino acids. The racemic mixture, rather than a single isomer, is provided to the animals as a feed supplement. When metabolizing the racemic mixture of α-hydroxy analogs of naturally occurring α-amino acids, the animals must convert the α-hydroxy analogs to the corresponding L-isomers of the α-amino acids before the isomer can be incorporated in protein synthesized by the animal.

In order to improve the nutrient utilization and energy of an animal, it is therefore desirable to provide the animal with the α-hydroxy analog isomer that can be most efficiently metabolized to the L-isomer. Thus, in a racemic mixture of D- and L-isomers of α-hydroxy analogs of naturally occurring α-amino acids, the D- and L-isomers must be resolved, or separated from one another and isolated. Once isolated, the isomer which is most efficiently utilized may be incorporated as a nutrient in an animal feed supplement. The isomer that is less-efficiently metabolized can subsequently be chemically converted to the more-efficiently metabolized isomer and be incorporated in an animal feed supplement.

While the isomer that is less-effectively metabolized into the L-isomer requires more energy from the animal, the isomer may be beneficially used in specific applications. One such application includes the use of the isomer as an appetite suppressants.

L(+)-lactic acid is widely used in the food and cosmetic industry. The L(+) form is the form which is present in the human body (e.g. skin, hair and muscles). Both the racemic mixtures and D(-) forms are less suitable for cosmetic applications because they are not as mild as the L(+) form.

L-Malic has excellent acidulant characteristics and finds uses in beverages and pharmaceuticals. It is an important natural organic acid, used as an additive in foodstuffs and is widely distributed in vegetables and fruits. It is used in the acids for wine, acid drink fruit juice, soda water and various soft drinks.

The supplementation of methionine to ruminant animals is more effectively accomplished by administering an isomer or an analog of L-amino acids because free methionine shows degradation by ruminal bacteria. 2-Hydroxy-4-(methylthio)butanoic acid (HMB) is an α-hydroxy analog of methionine, and is bioavailable and can be used as a feed supplement to chicken, pigs and ruminant animals. HMB is particularly efficacious with ruminant animals as compared to free methionine because HMB can resist ruminal degradation by microbes. The D and L-α-hydroxy analogs undergo enzymatic oxidation to produce an α-keto analog of methionine which is a limiting amino acid in most animals (i.e., it needs to be supplemented in the feed).

Amino acids and their hydroxy analogs as produced by chemical synthesis are ordinarily obtained as racemic mixtures, the D-enantiomer of which needs to be absorbed and then converted to L-enantiomer form being used by the animal.

U.S. Pat. No. 6,605,590 describes oligopeptides comprising amino acids that are end capped with an α-hydroxy carboxylic acid such as HMB acid. As disclosed in the patent, the hydroxy acid used for the end cap is preferably of the L-configuration. Similar preferences prevail for the purpose of providing or enhancing bioactivity in peptides developed for other human or animal nutritional or pharmaceutical uses.

Racemic mixtures of α-amino acids and their α-hydroxy analogs have been resolved by chromatographic separation of enantiomers. This process is time-consuming, expensive, and is unable to provide a product that contains an enriched concentration of the desired enantiomer in quantities that can cost-effectively be utilized as an animal feed supplement. As a result, feed supplements of α-hydroxy analogs of naturally occurring α-amino acids are often provided as a racemic mixture of both the D- and L-isomers. While the cost chromatographic separation may be justified for the isolation of enantiomers to be used in pharmaceutical applications, there is nonetheless a need for separation methods that are more efficient and more economical.

Enzymatic methods for the preparation of enantiopure malic and aspartic acid derivatives in organic solvents are described in Tetrahedron Asymmetry (1999), 10(22), 4405. A process for the preparation of D-lactic acid is described in Shie et al. TW 95-94105462 (2001). The manufacture of L-acetylmalic acid and D-malic acid using lipase is described in Maeda et al. JP 11056387.

A cost-effective method of resolving racemic mixtures of α-hydroxy analogs in sufficient quantities for use as animal feed supplements is therefore desirable.

### SUMMARY OF THE INVENTION

Among the various aspects of the present invention, therefore, is the provision of a new process for resolving a racemic mixture of α-hydroxy analog stereoisomers into mixtures containing an enantiomeric excess of one of the stereoisomers, the provision of a process for further resolving a mixture containing an enantiomeric excess of one of the stereoisomers, the provision of a continuous process of resolving a mixture containing an enantiomeric excess of one of the stereoisomers, and the provision of a feed supplement containing an enantiomeric excess of an α-hydroxy analog.

Briefly, therefore, the present invention is directed to a process for resolving an enantiomeric mixture of α-amino acid having the following formula: 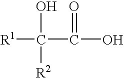
wherein R¹ is hydrogen, hydrocarbyl or substituted hydrocarbyl,
wherein R² is hydrogen, hydrocarbyl or substituted hydrocarbyl, and

When R¹ is a substituted hydrocarbyl comprising a phosphorus atom, R² is preferably hydrocarbyl or substituted hydrocarbyl. Substituted hydrocarbyl may include heteroatoms, i.e., the hydrocarbyl substituent may for example, include alkylthioalkyl, alkyldithioalkyl, alkoxylalkyl, sulfhydrylalkyl, hydroxy alkyl, and the like. The process comprises forming a reaction mixture comprising (i) an enantioselective enzyme, and (ii) an enantiomeric mixture of esters of the α-hydroxy acid or derivatives thereof, wherein the enantioselective enzyme preferentially hydrolyzes a first enantiomeric ester to produce a first α-hydroxy acid enantiomer corresponding to said first enantiomeric ester; forming a reaction product from the reaction mixture, the reaction product comprising (i) the first α-hydroxy acid enantiomer and (ii) unreacted α-hydroxy acid ester; separating the first α-hydroxy acid enantiomer and unreacted α-hydroxy acid ester from each other.

The present invention is also directed to a stereoisomeric mixture having an enantiomeric excess of a stereoisomer of an α-hydroxy acid having the following formula: 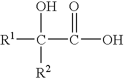
wherein R¹ and R² are as defined above. The stereoisomer mixture is formed by a process that comprises forming a reaction mixture comprising (i) an enantioselective enzyme, and (ii) an enantiomeric mixture of esters of the α-hydroxy acid, wherein the enantioselective enzyme preferentially hydrolyzes a first enantiomeric ester to produce a first mixture of α-hydroxy acid enantiomers that contains an enantiomeric excess of the first α-hydroxy acid enantiomer; forming a reaction product from the reaction mixture comprising (i) the first mixture of α-hydroxy acid enantiomers, and (ii) unreacted second α-hydroxy acid ester; and separating the first mixture of α-hydroxy acid enantiomers and unreacted α-hydroxy acid ester enantiomers in the reaction product from each other. The first mixture may be optionally further processed to enhance the relative proportion of a desired isomer, isolate the desired isomer, e.g., by chromatography or other means, or otherwise refine the mixture.

The present invention is also directed to a feed supplement comprising the above-described stereoisomeric mixture and a process of feeding the stereoisomeric mixture to an animal.

The present invention is further directed to the preparation of enantiomerically purified, or enantiomerically enhanced, α-hydroxy acids which are useful as intermediates in the preparation of nutritional and pharmaceutical products for animal or human application.

The present invention is also directed to a process for producing and resolving an α-hydroxy acid enantiomer or derivative thereof in an enantiomeric mixture. The process comprises forming a first reaction mixture comprising an α-hydroxy acid and an alcohol; forming a first product mixture from the first reaction mixture, the first product mixture comprising an α-hydroxy ester corresponding to the α-hydroxy acid; forming a second reaction mixture from the first product mixture, the second reaction mixture comprising the α-hydroxy ester and an enantioselective enzyme; forming a second product mixture from the second reaction mixture, the second product mixture comprising a first α-hydroxy acid and unhydrolyzed α-hydroxy ester, wherein the first α-hydroxy acid is produced by the enantioselective hydrolysis of a first enantiomer of the α-hydroxy ester; and separating the α-hydroxy acid produced by the enantioselective hydrolysis of the α-hydroxy ester and the unhydrolyzed α-hydroxy ester in the second product mixture from each other.

The present invention is also directed to a process for producing and continuously resolving an α-hydroxy acid enantiomer or derivative thereof in an enantiomeric mixture. The process comprises forming a first reaction mixture comprising an α-hydroxy acid and an alcohol; forming a first product mixture from the first reaction mixture, the first product mixture comprising an α-hydroxy ester corresponding to the α-hydroxy acid; forming a second reaction mixture from the first product mixture, the-second reaction mixture comprising the α-hydroxy ester; forming a second product mixture by continuously contacting the second reaction mixture with an immobilized enantioselective enzyme, the second product mixture comprising a first α-hydroxy acid and unhydrolyzed α-hydroxy ester, wherein the first α-hydroxy acid is produced by the enantioselective hydrolysis of a first enantiomer of the α-hydroxy ester; and continuously separating the α-hydroxy acid produced by the enantioselective hydrolysis of the α-hydroxy ester from the unreacted α-hydroxy ester.

Other aspects and features of this invention will be in part apparent and in part pointed out hereinafter. I

### BRIEF DESCRIPTION OF THE DRAWINGS

FIG. 1 is a flowchart of the experimental protocol followed in Examples 1 to 5.

FIG. 2A is a chromatogram illustrating the separation of HMB acid and HMB-methyl ester.

FIG. 2B is a chromatogram illustrating the separation of HMB acid and HMB-ethyl ester.

FIG. 2C is a chromatogram illustrating the separation of HMB acid and HMB-propyl ester.

FIG. 2D is a chromatogram illustrating the separation of HMB acid and HMB-butyl ester.

FIG. 3A is a RPLC chromatogram illustrating the separation of HMB-methyl ester after non-enzymatic hydrolysis.

FIG. 3B is a RPLC chromatogram illustrating the separation of HMB-methyl ester after lipase-catalyzed hydrolysis wherein the lipase is obtained from Candida rugosa.

FIG. 3C is a RPLC chromatogram illustrating the separation of HMB-methyl ester after lipase-catalyzed hydrolysis wherein the lipase is Novo 435.

FIG. 3D is a RPLC chromatogram illustrating the separation of HMB-methyl ester after lipase-catalyzed hydrolysis wherein the lipase is obtained from Aspergillus niger.

FIG. 3E is a RPLC chromatogram illustrating the separation of HMB-methyl ester after lipase-catalyzed hydrolysis wherein the lipase is porcine pancreatic lipase (PPL).

FIG. 4A is a RPLC chromatogram illustrating the separation of HMB-butyl ester after non-enzymatic hydrolysis.

FIG. 4B is a RPLC chromatogram illustrating the separation of HMB-butyl ester after lipase-catalyzed hydrolysis wherein the lipase is obtained from Candida rugosa.

FIG. 4C is a RPLC chromatogram illustrating the separation of HMB-butyl ester after lipase-catalyzed hydrolysis wherein the lipase is Novo 435.

FIG. 4D is a RPLC chromatogram illustrating the separation of HMB-butyl ester after lipase-catalyzed hydrolysis wherein the lipase is obtained from Aspergillus niger.

FIG. 4E is a RPLC chromatogram illustrating the separation of HMB-butyl ester after lipase-catalyzed hydrolysis wherein the lipase is porcine pancreatic lipase (PPL).

FIG. 5A is a RPLC chromatogram illustrating the separation of HMB-ethyl ester after non-enzymatic hydrolysis.

FIG. 5B is a RPLC chromatogram illustrating the separation of HMB-ethyl ester after lipase-catalyzed hydrolysis wherein the lipase is obtained from Candida rugosa.

FIG. 5C is a RPLC chromatogram illustrating the separation of HMB-ethyl ester after lipase-catalyzed hydrolysis wherein the lipase is Novo 435.

FIG. 5D is a RPLC chromatogram illustrating the separation of HMB-ethyl ester after lipase-catalyzed hydrolysis wherein the lipase is obtained from Aspergillus niger.

FIG. 5E is a RPLC chromatogram illustrating the separation of HMB-ethyl ester after lipase-catalyzed hydrolysis wherein the lipase is porcine pancreatic lipase (PPL).

FIG. 6A is a RPLC chromatogram illustrating the separation of HMB-propyl ester after non-enzymatic hydrolysis.

FIG. 6B is a RPLC chromatogram illustrating the separation of HMB-propyl ester after lipase-catalyzed hydrolysis wherein the lipase is obtained from Candida rugosa.

FIG. 6C is a RPLC chromatogram illustrating the separation of HMB-propyl ester after lipase-catalyzed hydrolysis wherein the lipase is Novo 435.

FIG. 6D is a RPLC chromatogram illustrating the separation of HMB-propyl ester after lipase-catalyzed hydrolysis wherein the lipase is obtained from Aspergillus niger.

FIG. 6E is a RPLC chromatogram illustrating the separation of HMB-propyl ester after lipase-catalyzed hydrolysis wherein the lipase is porcine pancreatic lipase (PPL).

FIG. 7A is a chromatogram illustrating the separation of HMB enantiomers obtained after non-enzymatic hydrolysis of HMB-butyl ester.

FIG. 7B is a chromatogram illustrating the separation of HMB enantiomers obtained after enzymatic hydrolysis of HMB-butyl ester wherein the enzyme is the lipase is porcine pancreatic lipase (PPL).

FIG. 8A is a chromatogram illustrating the separation of HMB enantiomers obtained after non-enzymatic hydrolysis of HMB-propyl ester.

FIG. 8B is a chromatogram illustrating the separation of HMB enantiomers obtained after enzymatic hydrolysis of HMB-propyl ester wherein the enzyme is the lipase is porcine pancreatic lipase (PPL).

FIG. 9A is a chromatogram illustrating the separation of HMB-propyl ester enantiomers obtained after non-enzymatic hydrolysis.

FIG. 9B is a chromatogram illustrating the separation of HMB-propyl ester enantiomers obtained after enzymatic hydrolysis wherein the enzyme is the lipase is porcine pancreatic lipase (PPL).

FIG. 10A is a chromatogram illustrating the separation of HMB-butyl ester enantiomers obtained after non-enzymatic hydrolysis.

FIG. 10B is a chromatogram illustrating the separation of HMB-butyl ester enantiomers obtained after enzymatic hydrolysis wherein the enzyme is the lipase is porcine pancreatic lipase (PPL).

FIG. 11 is a chromatogram of the n-propyl ester product obtained in the preparation of Example 7.

FIG. 12A is a chromatogram of the product obtained after exposure of the ester product of Example 7 to free PPL enzyme at room temperature for 2 hours as described in Example 8.

FIG. 12B is a chromatogram of the product obtained after exposure of the ester product of Example 7 to free PPL enzyme at room temperature for 3.30 hours as described in Example 8.

FIG. 13A is a chromatogram of the product obtained according to Example 8 after exposure of the ester product of Example 7 to PPL at room temperature for 2 hours, wherein the PPL enzyme is immobilized on alginate beads.

FIG. 13B is a chromatogram of the product obtained according to Example 8 after exposure of the ester product of Example 7 to PPL at room temperature for 3.30 hours, wherein the PPL enzyme is immobilized on alginate beads.

FIG. 14A is a chromatogram similar to that of FIG. 13A, except that hydrolysis in the presence of PPL immobilized on alginate beads has been conducted at a temperature of 37° C.

FIG. 14B is a chromatogram similar to that of FIG. 13B, except that hydrolysis in the presence of PPL immobilized on alginate beads has been conducted at a temperature of 37° C.

FIG. 15A is a chromatogram obtained according to Example 8 after exposure of the ester product of Example 7 to alginate beads free of enzyme at a temperature of 37° C. for 2 hours.

FIG. 15B is a chromatogram similar to that of FIG. 15A but is taken after 3.30 hours rather than 2 hours exposure to the alginate beads at 37° C.; and

FIG. 16 is a plot of free acid to ester ratio after exposure of the ester product of Example 7 to either gel-immobilized PPL enzyme at room temperature, gel-immobilized PPL enzyme at 37° C. or free enzyme at 37° C.

### DETAILED DESCRIPTION OF THE PREFERRED EMBODIMENTS

The present invention relates to a process for resolving an enantiomeric mixture of α-hydroxy acids or derivatives thereof. In accordance with the present invention, it has been discovered that an enantiomeric mixture of an α-hydroxy acid or derivative thereof can be resolved through esterification of the α-hydroxy acid or derivative and enzymatically hydrolyzing the ester (hereinafter interchangeably referred to as “ester of the α-hydroxy acid” or “α-hydroxy acid ester”) wherein one enantiomeric ester is preferentially hydrolyzed to its corresponding acid. The acid and non-hydrolyzed ester are separated into enantiomerically enriched mixtures of D- and L-isomers. The resolving process can be repeated using the enantiomerically enriched mixtures to further increase the concentration of one isomer over the other. The isomers present in the enantiomerically enriched mixtures are isolated to obtain a mixture comprising D- and L-isomers of α-hydroxy acids or derivatives wherein the mixture contains an enantiomeric excess of one stereoisomer.

The process of the invention is especially advantageous for the separation of D- and L-isomers of 2-hydroxy-4-methylthiobutanoic acid or the hydroxy analog of lysine. It is also applicable to the separation of the enantiomers of other α-hydroxy acids. Other acids of particular value which may be separated in accordance with the invention include the hydroxy acid analogs of naturally occurring essential amino acids, i.e., isoleucine, phenylalanine, leucine, threonine, tryptophan, histidine and valine. Enantiomers of these hydroxy acids may be uniquely advantageous as intermediates in the preparation of pharmaceutical peptides, other pharmaceutical compounds or animal feed formulations.

Because chemical synthesis alternatives are generally impractical and commercially unproven for the preparation of substantially pure HMB enantiomers, chemical synthesis of even one gram of pure HMB enantiomer can be quite expensive. In accordance with the present invention a batch-scale process has been developed for the enzymatic enantioenrichment of HMB. Of the enantiomerically selective enzymes that may be used in the process of the invention, lipases are perhaps the most popular enzymes in biocatalysis because they couple a wide specificity to a high regio and enantioselectivity and specificity, therefore, may be used in many different reactions. Four lipases: Candida rugosa, candida antartica, aspergillus niger and porcine pancreatic lipase (PPL) have been used in confirming the ester hydrolysis yield obtainable from HMB methyl ester (HMBME), HMB ethyl ester (HMBEE), HMB propyl ester (HMBPE), HMB butyl ester (HMBBE) and HMB isopropyl ester.

Although a variety of combinations of enzyme and α-hydroxy acid ester may be used in the process of the invention, certain particular advantages have been observed where the ester hydrolysis of HMB-propyl ester is carried out using PPL enzyme. HMB-propyl ester is among the more suitable substrates because it is not susceptible to chemical hydrolysis (lesser background chemical hydrolysis) and higher enzyme selectivity.

As described below, improvements in the implementation of the process of the invention have been achieved by immobilizing the hydrolytic enzyme on a solid support, such as, for example, polysaccharide gel beads obtained by cross-linking an alginate with an aldehyde, and gelling with the addition of a divalent metal ion such as Ca⁺⁺. It has further been discovered that the implementation of the process may be extended to a continuous selective hydrolysis process, as carried out, for example in a fixed bed reactor system comprising a catalyst bed containing enantiomerically selective enzyme immobilized on polysaccharide gel beads.

Free enzymes dissolved or dispersed in a liquid reaction medium may be used in the process of the invention to catalyze the enantiomerically selective hydrolysis.

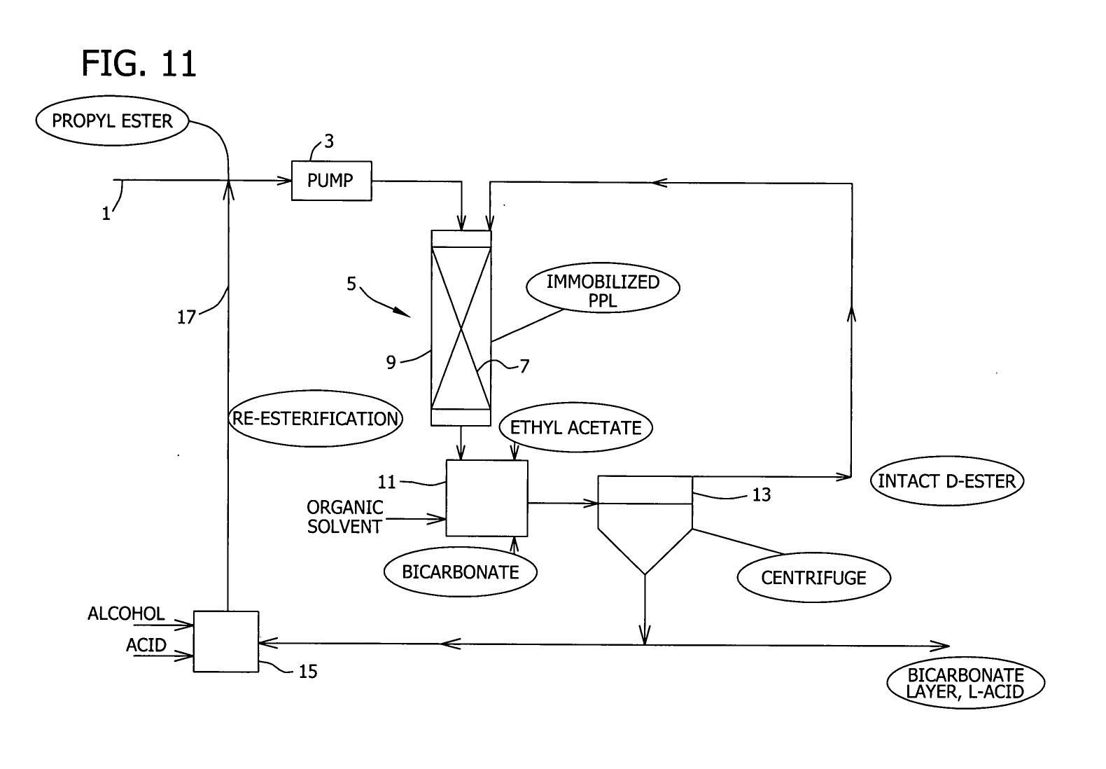

However, in various preferred embodiments, the catalyst is not freely dispersed in the liquid medium but is instead immobilized on a solid support. Immobilization of the enzyme on a solid support facilitates separation of the liquid product mixture from the enzyme and use of the enzyme through a plurality of batch reaction cycles. In accordance with such embodiments of the invention, the catalyst may be immobilized, for example, on solid pellets or beads which can be used, not only in batch hydrolysis, but also in a continuous hydrolysis reaction system. For example, the catalyst beads may be formed into a fixed or fluidized bed for a tubular reactor through which a reaction mixture comprising the α-hydroxy acid and liquid medium are caused to pass in essentially plug flow. A continuous fixed bed reaction system of this nature is depicted in FIG. 11 and described in further detail below.

It has been found that an enantiomerically selective hydrolytic enzyme, such as lipase, may be conveniently and advantageously immobilized on a hydrogel support comprising, for example, a polysaccharide derived from an alginate salt. Other polysaccharides may also be used. It has further been found that a support of particularly suitable properties is obtained from combining an alkali metal polysaccharide such as sodium alginate with a protein such as gelatin, then exchanging the alkali metal ions for divalent metal ions, typically Ca, Mg, Mn or Zn. A support of this nature is described by Fadnavis et al., “Gelatin Blends with Alginate: Gels for Lipase Immobilization and Purification,” Biotechnol. Prog., 2003, 19, 557-564. As described by Fadnavis et al., it is believed that the carboxylic acid functions of the alginate interact with the amine functions of the gelatin to form a complex in which the enzyme is entrapped. As further discussed by Fadnavis, the alginate or other polysaccharide and the gelatin or other protein are preferably also cross-linked with glutaraldehyde. Cross-linking through the dialdehyde lends further stability to the support structure.

The support may be formed as porous beads containing the entrapped enzyme. The α-hydroxy acid ester substrate gains access to the enzyme by diffusion through the pores of the catalyst bead, with product L-acid and D-ester (or D-acid and L-ester) being removed from the bead by diffusion along a path through the same porous structure.

Preferably the porous beads contain between about 0.1 and about 10 wt. %, preferably between about 0.2 and about 3 wt. %, more preferably between about 0.5 and about 2 wt. % enzyme. For hydrolysis of esters which are not highly hydrophobic, and especially for use in a fixed bed tubular reactor, the beads preferably have a diameter or principal dimension between about 1 and about 4 mm, more typically between-about 1.5 and about 3.5 mm, most typically about 2 to about 2.5 mm. For more hydrophobic substrates, it may be preferable to use smaller beads, for example in the range of about 0.2 to about 1.5 mm, or more typically 0.3 to 0.75 mm. Depending on the application, however, the size of the beads or particles can range from perhaps as little as 0.05 mm to 10 or 20 mm. Alternatively, the support may be formed as a relatively large monolithic structure comprising open pores through which the reaction mixture can percolate.

Polysaccharide-protein beads containing entrapped hydrolytic enzymes are prepared by combining the polysaccharide, protein and enzyme, and contacting them in an aqueous medium with a divalent cation, preferably calcium, but optionally also Ba, Zn or Mg. Interaction of the polysaccharide and protein produces a gel which is solidified by reaction with the divalent cation. Optionally, the polysaccharide and protein can be cross-linked with a dialdehyde such as glutaraldehyde, 1,6-dihexanal or 1,4-dibutanal prior to or simultaneously with contact of the polysaccharide/protein mixture with divalent cation.

In a preferred embodiment of the invention, beads comprising entrapped enzyme may be prepared in the manner generally described by Fadnavis. An aqueous premixture is prepared comprising a solution or dispersion of an alkali metal salt of the polysaccharide and, preferably, a protein, together with the enzyme to be supported. In the case of Na alginate and gelatin, the premixture may typically contain alginate up to the saturation level of about 5 wt. % and gelatin in a weight ratio to the alginate between about 0.2:1 and about 1.5:1. A preferred composition contains about 5 wt. % Na alginate and about 3 wt. % gelatin. Hydrolytic enzyme such as a lipase is incorporated into the solution in a proportion that is typically between about 5% and about 25% based on the sum of polysaccharide and protein. Optionally but preferably, a dialdehyde such as glutaraldehyde is incorporated in proportion effective to retain the enzyme within the bead but not to create cross-linking so dense as to impair diffusion of ester substrate to the enzyme sites, or deactivate the enzyme. Those skilled in the art can readily determine the appropriate dialdehyde content for a particular application. In a number of applications, a molar ratio to the saccharide repeating units between about 0.004 and about 0.01, e.g., in a weight ratio to a polysaccharide such as Na alginate between about 0.02% and about 0.5%, more typically between about 0.05% and about 0.2%.

After the premixture is initially prepared, it is preferably allowed to stand at ambient temperature for a period of time, typically 30 to 120 minutes. Thereafter it is heated, e.g., to a temperature of at least about 90° C., typically, 1000 to 140° C. for a relatively short period, e.g., 10 to 30 minutes. Processing the premixture according such time/temperature schedule results in formation of a polysaccharide/protein complex which is crosslinked via the dialdehyde, with the enzyme trapped in the polysaccharide/protein matrix. The premixture is thereby converted to a gel which is preferably cooled back to ambient temperature. At 150 to 40° C., the gel has a viscosity suitable for formation of drops from which the catalyst support beads can be prepared by contact with divalent cation. For example, the viscosity at 25° C. of a gel prepared in the manner described above may be especially well suited to the formation of coherent drops of gel that may be contacted with divalent cation to yield beads in which the enzymes are entrapped.

In accordance with a preferred method of preparation, the aqueous premixture is passed through a fine aperture, e.g., via a syringe comprising a fine needle to form a coherent drop, the drop is disengaged from the aperture and introduced into an aqueous bead formation medium wherein it is contacted with divalent cation. The aqueous bead formation medium preferably contains between about 0.05 and about 0.3 moles/liter, more typically between about 0.1 and about 0.25 moles/liter divalent cation. Typically, the bead formation medium is spaced by a suitable distance from the point of release of the drop from the aperture. Conveniently, the aperture is located vertically above the bead formation medium so that the bead is transported by gravity from the release point to the medium. Alternatively, the drop may be transported in an air or gas stream. Where the aperture is transported by gravity, a suitable distance is sufficient to allow the drop to conform to a substantially spherical shape, without accelerating it to a velocity which will cause it to disintegrate on impact with the bead formation medium. For these purposes, the distance may be in the range of 2 cm to 100 cm, preferably between about 5 and about 50 cm. A distance between about 5 and about 30 cm, or more preferably 5 cm to 20 cm, has been found particularly convenient.

The gel beads are found to solidify immediately on contact with an aqueous bead formation medium, and settle from the bead formation medium. To assist in preserving integrity of the drops as they impact the surface and react with the divalent metal ions, the bead formation medium is preferably maintained at a relatively low temperature, e.g., in the range of −10° to 15° C., preferably less than about 5° C. By maintaining the aqueous bead formation medium in the cold, the viscosity of the gel is typically maintained at a level which preserves the integrity of the drop as it impacts on and settles through the bead formation medium. To assist in drop formation, the gel is preferably delivered to the aperture at a temperature 10 to 40 Centigrade degrees higher than the temperature of the bead formation medium, most typically at ambient temperature, i.e., 15° to 40° C., more typically 25° to 30° C. To enhance binding of enzymes to the carboxylic acid sites within the pores of the catalyst bead, the bead formation medium is preferably maintained at a pH lower than about 7.5, more preferably lower than about 7.0, more preferably in a range between about 4 and about 6. The size of the beads can be controlled by selection of the size of the orifice. Those skilled in the art can readily select an aperture size appropriate for a particular gel formulation. Generally, it has been found that a standard hypodermic needle is effective in producing drops of a size appropriate for producing beads having a diameter in the aforesaid ranges If desired, the size of beads can be reduced by milling to yield catalyst particles of relatively small diameter, e.g., 0.25 to 0.75 mm, which may be suitable for hydrolysis of relatively hydrophobic ester substrates.

After a batch of catalyst beads has been formed by introduction of a series of drops into the medium, the beads are separated from the medium and washed several times with water for removal of unbound enzyme from the bead surfaces. The beads are then ready for use in the selective hydrolysis process of the invention.

The present invention also relates to purified α-hydroxy acids or derivatives and methods of use thereof.

α-Hydroxy Acids

Enantiomeric mixtures of a variety of α-hydroxy acids can be resolved according to embodiments of the present invention.

In one embodiment, the present invention includes a process for resolving a racemic mixture of α-hydroxy acids having the following formula: 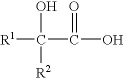
wherein R¹ is hydrogen, hydrocarbyl or substituted hydrocarbyl and wherein R² is hydrogen, hydrocarbyl or substituted hydrocarbyl. Substituted hydrocarbyl substituents which may constitute R¹ or R² include sulfur-containing substituents such as methylthioethyl (as in the hydroxy analog of methionine) and HS—CH₂— (hydroxy analog of cysteine), as well as substituents derived from the hydroxy analogs of other sulfur-containing amino acids such as lysine or cystine, or from aralkyl, aryl or heteroaryl substituted acids such as the hydroxy analogs of tryptophan and phenylalanine. A further listing of useful amino acid analogs is set forth hereinbelow. Where R¹ is a substituted hydrocarbyl comprising a phosphorus atom, R² is preferably a hydrocarbyl or substituted hydrocarbyl.

In various particular embodiments, the present invention includes a process for resolving a racemic mixture of α-hydroxy acids wherein the α-hydroxy acid residue is the residue of any of the following naturally occurring α-hydroxy acids: benzilic acid, citric acid, glyceric acid, glycolic acid, glyoxylic acid, lactic acid, malic acid, mandelic acid, pyruvic acid, and tartaric acid.

In other embodiments, the α-hydroxy acid residue may be the α-hydroxy acid analog of a naturally occurring α-amino acid. The α-hydroxy amino residue(s) may be the residue(s) of any of the naturally occurring α-amino acids, e.g., asparagine, alanine, valine, leucine, isoleucine, phenylalanine, proline, serine, threonine, cysteine, methionine, tryptophan, tyrosine, glutamine, aspartic acid, glutamic acid, lysine, arginine, and histidine.

In certain preferred embodiments, the α-hydroxy acid residue may be an α-hydroxy acid analog of an essential α-amino acid. The α-amino acid residue(s) include the residue(s) of one or more essential α-amino acids, i.e., isoleucine, phenylalanine, leucine, lysine, methionine, threonine, tryptophan, histidine and valine.

The novel process may provide a commercially attractive alternative for resolving enantiomeric mixtures of the α-hydroxy acid analog of methionine and/or lysine.

The α-hydroxy acid analog of methionine, i.e., 2-hydroxy-4-(methylthio)butyric acid, also commonly referred to as methionine hydroxy analog, HMB, or HMBA, is commercially available in 85-90 wt. % aqueous solution under the trade designation ALIMET® from Novus International, Inc., St. Louis, Mo.

Enantioselective Enzymes

Enzymes that may be used in the process according to the present invention include enzymes that preferentially hydrolyze ester bonds of one enantiomer of an enantiomeric mixture of α-hydroxy acid esters. For example, an enzyme can be used in the process of the present invention if, when mixed with a racemic mixture of an ester of an α-hydroxy acid, it preferentially hydrolyzes the L-isomer of the α-hydroxy acid ester. Alternatively, the enzyme can be used if it preferentially hydrolyzes the D-isomer ester. After preferential hydrolysis has occurred, the resulting enantiomeric mixture comprises a mixture of the α-hydroxy acid and the ester of the α-hydroxy acid wherein one stereoisomer is more predominant in the hydrolyzed isomers of the α-hydroxy acid. Conversely, the stereoisomer that is not preferentially hydrolyzed is more predominant in the non-hydrolyzed enantiomers of the α-hydroxy acid ester. In biological systems, natural enzymes that exhibit a stereospecific preference will typically catalyze reactions on the L-isomer of a compound such as the L-isomer of α-amino acids.

In one embodiment, the enantioselective enzyme includes a lipase enzyme. Examples of representative lipase enzymes for use in the process of the present invention include porcine pancreatic (“PPL”), lipase obtained from Candida rugosa, lipase obtained from Aspergillus niger, and combinations thereof. These enzymes have been found to preferentially hydrolyze the L-isomer of esters of α-hydroxy acids.

Esters of α-Hydroxy Acid

In accordance with the present invention, the α-hydroxy acid that is to be enzymatically resolved must first be in the form of an ester. Esterification of α-hydroxy acids may be performed according to an esterification process known in the art.

In one embodiment, esterification of the α-hydroxy acid is performed by mixing an enantiomeric mixture of the α-hydroxy acid with an alcohol. The mixture is acidified and permitted to react for a period of time, e.g., overnight, resulting in the formation of an enantiomeric mixture of esters of the α-hydroxy acid. Once formed, the esters of the α-hydroxy acid are recovered. Recovery may be performed, for example, by removing the excess alcohol with a vacuum, rotary evaporator, and the like.

In general, the esterification reaction may be carried out over a relatively wide range of temperatures, e.g., at a temperatures of least 65° C., typically between about 65° C. and about 95° C., more typically between about 70° C. to about 90° C., preferably between about 80° C. to about 90° C.

It is desirable to select an alcohol that will react with the α-hydroxy acids to form a substantial yield of an ester product. Alcohols which form esters of α-hydroxy acids which undergo minimal non-enzymatic hydrolysis in an aqueous solution are particularly useful in the process of the present invention as a non-enzymatic hydrolysis process will typically not be enantioselective in nature. The reversible esterification/hydrolysis reaction is illustrated as follows: 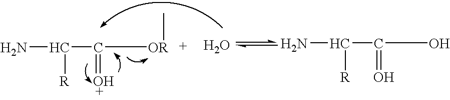
wherein R is hydrocarbyl or substituted hydrocarbyl.

Examples of alcohols that may be used in the esterification process include alkyl, alkenyl, and alkynyl alcohols. Typically, the alcohol may be an alkyl alcohol, preferably selected from lower alcohols having 1-20 carbons. Specific exemplary alcohols include methyl, ethyl, propyl, isopropyl, butyl, tert-butyl, pentyl, hexyl, heptyl, octyl, nonyl, and decyl alcohols. For some applications, propyl and butyl alcohols may be particularly preferred based on considerations of selectivity and cost. For the separation of L- from D-isomers of HMBA, the n-propyl ester is susceptible to very highly selective hydrolysis as catalyzed by an enantiomerically selective enzyme such as lipase. Other esters in the C₂ to C₈ range are also suitable for separating enantiomers of HMBA and other α-hydroxy acids.

Hydrolysis Process

The ester of the α-hydroxy acid is enantioselectively hydrolyzed according to the process of the present invention by combining the ester of the α-hydroxy acid and an enantiomerically selective hydrolytic enzyme, such as a lipase, in the presence of water to form a reaction mixture. Preferably, water is present in excess to drive the hydrolysis reaction to completion. For example, the reaction mixture preferably comprises at least about 10 moles water per mole ester, more preferably at least about 100 moles water per mole ester, typically between about 100 and about 1000 moles water per mole ester. In such relative proportions esters of HMBA and lower alcohols are generally soluble in the aqueous phase.

The pH of the reaction mixture is adjusted to be at least about 5. Typically, the pH may be adjusted to be between about 5 and about 9, for example, between about 6 and about 8, conveniently between about 6.5 and about 7.5. Preferably, the reaction mixture includes a buffer, such as, e.g., tris(hydroxymethyl)aminomethane hydrochloride, or alkali metal phosphate, which maintains the pH in the desired range despite the progressive increase in the concentration of α-hydroxy carboxylic acid in the reaction mixture as the reaction proceeds.

In general, the reaction may be carried out over a relatively wide range of temperatures, e.g., at least 15° C. For example, the temperature range may be between about 15° C. to about 35° C., conveniently in the typically ambient range between about 20° C. to about 30° C. Room temperature hydrolysis reaction is ordinarily quite satisfactory. Reaction under mild temperature and pH conditions helps to minimize chemical hydrolysis that could otherwise compromise selectivity.

Reaction time may vary widely. Generally a time within the range of 1 hour to 48 hours is sufficient for the reaction. Usually, the reaction can be carried to satisfactory conversion in between about 2 and about 24 hours, more typically between about 4 and about 18 hours. In laboratory or small scale manufacturing operations, the reaction can often be conducted most conveniently by running to substantial completion overnight.

The reaction may be conducted with/without mechanical or other physical agitation. To ensure adequate contact between the lipase and the ester of the α-hydroxy acid the reaction mixture may be subjected to periodic or continuous agitation by stirring the reaction mixture during the course of the reaction.

After a period of time has elapsed for the esters of the α-hydroxy acid to react, the reaction mixture solvents may be removed leaving a dry product comprising hydrolyzed isomers α-hydroxy acid and non-hydrolyzed isomers of the α-hydroxy acid ester.

In one embodiment, the dry product is dissolved in a solvent, e.g., an alcohol such as ethanol, and filtered to form a filtrate. The solvent may then be removed from the filtrate forming a filtered dry product.

The dry product or filtered dry product may be contacted with a mixture of organic and aqueous solvents wherein the organic and aqueous solvents are immiscible and form an aqueous phase and an organic phase. The isomers of the α-hydroxy acid preferentially migrate to the aqueous phase and the isomers of the α-hydroxy acid ester preferentially migrate to the organic phase, with the result that the α-hydroxy acid is substantially separated from the α-hydroxy acid esters by phase (liquid/liquid) extraction.

In general, the organic phase comprises an organic solvent selected from the group consisting of alkanes, alkenes, aryls and suitable derivatives thereof. For example, the organic solvent may be selected from the group consisting of moderately polar solvents which are not miscible with water, short chain esters such as ethyl acetate, propyl acetate or longer. The aqueous phase is substantially immiscible in the selected organic solvent. Examples of aqueous phase compositions include water, sodium bicarbonate solution, potassium bicarbonate solution, calcium bicarbonate solution, and the like.

The phases are separated from each other and the isomers of the α-hydroxy acid and the ester isomers of the α-hydroxy acid contained in each phase are further isolated from their respective phases to form enantiomerically enriched mixtures, each comprising an enantiomeric excess of a stereoisomer. Where the aqueous extract contains primarily L-acid, the organic phase comprises a relatively higher proportion of D-isomer, and vice versa. Isolation of α-hydroxy acid isomers from the aqueous phase and the ester isomers of the α-hydroxy acid from the organic solvent phase may be effected through evaporation, rotary evaporation, vacuum distillation, steam distillation, evaporative crystallization, freeze-drying or similar means.

Once isolated, the enantiomerically enriched mixtures may be analyzed to determine the concentrations of stereoisomers contained in each mixture. This may be performed by liquid chromatography or similar analytical means known in the art.

The enantiomerically enriched mixtures may be further enriched by repeating the above process. The enantiomerically enriched mixtures containing an enantiomeric excess of D-isomers of the α-hydroxy acid ester may be hydrolyzed to form a mixture comprising an enantiomeric excess of the D-isomer of the α-hydroxy acid.

Alternatively, the enantiomerically enriched mixtures containing an enantiomeric excess of D-isomers of the α-hydroxy acid ester may be subjected to base racemization and the resulting racemic mixture reused as reactants in the above hydrolysis process to further isolate the L-isomer from the D-isomer.

The enantiomerically enriched mixtures containing an enantiomeric excess of L-isomers of the α-hydroxy acid may be further resolved by esterifying the α-hydroxy acid isomer product and subjecting the ester isomers to a further cycle of enzymatically selective hydrolysis according to the process described above to isolate another fraction of L-acid from D-ester.

Enzymes such as those described above are typically effective for quantitatively selective hydrolysis, i.e., to convert only L-ester or only D-ester to the corresponding acid. In actual practice, selectivity may nonetheless fall short of 100% due to competitive chemical hydrolysis. Chemical hydrolysis is ordinarily entirely non-selective, with the consequence that an enzymatic hydrolysis run to high conversion may be materially compromised by any significant chemical hydrolysis which acts on the pool of unreacted ester, and therefore may disproportionately convert the ester of the undesired isomer to the acid.

Under the conditions disclosed herein, the extent of chemical hydrolysis can be minimized. Typically, the selectivity of the enzymatic hydrolysis is effective to produce an α-hydroxy acid component of the product mixture which comprises at least 60% of one enantiomer, usually the L-acid. By proper selection of hydrolytic enzyme, ester substrate and reaction conditions, it has been proven feasible to obtain a product mixture in which the α-hydroxy acid component comprises at least 70% L-acid, or at least 80% L-acid, or even greater than 90% L-acid. For example, where an ester such as the n-propyl ester of HMBA is enzymatically hydrolyzed with PPL in an aqueous system at an initial ester concentration in the range between about 0.1 mM and the saturation concentration of the ester at a temperature and pH in the aforesaid range over a period of 12 to 36 hours, substantially 100% selectivity of L-ester to L-acid can be achieved, substantially independently of the conversion. Under such process conditions, competitive non-selective chemical hydrolysis may be negligible. In such instance, if the process is run to 50% conversion of ester, a product mixture is obtained in which the α-hydroxy acid component approaches 100% L-isomer, and the unreacted ester component approaches 100% D-isomer.

Where the selectivity is significantly less than 100%, either due to competitive chemical hydrolysis or less than 100% selective enzymatic hydrolysis, the fraction of desired stereoisomer may tend to decline with conversion. In such instances, the enzymatic hydrolysis may optionally be run to less than 50% conversion to obtain an acid fraction of a desired degree of enrichment, with a relatively lesser degree of enrichment of the remaining ester fraction with regard to the complementary isomer.

Where selectivity is significantly less than 100%, the process may optionally be run through a series of esterification, hydrolysis and separation cycles in order to enhance the proportion of a given stereoisomer in the ultimate acid product. Each cycle produces an enantiomerically enriched α-hydroxy acid, which may be re-esterified and subject to enantiomerically selective hydrolysis to further upgrade the desired isomer content. For example, if 60% selectivity is achieved in one cycle, a selectivity over 90% can be achieved in three cycles. Where the process is operated through a plurality of cycles, care must be taken to avoid racemization during the re-esterification, which may require the use of milder pH and temperature conditions than would otherwise be optimal for preparation of the ester. Plural esterification and enzymatic hydrolysis cycles may be appropriate, e.g., in the relatively low volume preparation of high assay L- or D-enantiomer of an expensive α-hydroxy acid.

Where esterification, hydrolysis and re-esterification are taken through multiple sequential cycles, each cycle also produces an enantiomerically enriched residual ester fraction, which can optionally be recycled to the preceding cycle in a fractional separation scheme which yields both the desired acid stereoisomer at one terminal and the complementary ester isomer at the other.

Continuous Enantioselective Process

In accordance with the present invention, the enantioselective hydrolysis of esters of α-hydroxy acids may proceed on a continuous basis. In such a process, a mixture comprising an ester of an α-hydroxy acid is continuously contacted with an immobilized enzyme of the type described above. The immobilized enzyme may be contained in any of a variety of types of vessels or containers generally known to those skilled in the art.

The continuous hydrolysis begins by forming a first reaction mixture comprising an α-hydroxy acid and an alcohol to produce a first reaction product containing the ester of the α-hydroxy acid. The α-hydroxy acid ester is then continuously or intermittently introduced to the immobilized enzyme within a reaction zone in order to enantioselectively hydrolyze the ester to produce a second reaction product comprising a mixture of enantiomers of the corresponding α-hydroxy acid and unreacted α-hydroxy ester. The α-hydroxy acid produced by the hydrolysis and unreacted α-hydroxy ester may be removed from the reaction zone continuously or intermittently.

FIG. 11 comprises a block flow diagram illustrating an embodiment of a continuous enzymatically selective hydrolysis process. An aqueous composition comprising an enantiomeric ester mixture is delivered from a supply 1 and optionally mixed with partially enriched L-isomer stream 17 which has been re-esterified and recycled. The aqueous ester mixture is transferred by a pump 3 to an enzymatic hydrolysis reactor 5. Reactor 5 comprises a fixed or fluidized bed 7 of granular or pelleted catalyst bodies within a tubular reactor vessel (column) 9. The catalyst bodies comprise the hydrolytic enzyme immobilized on a support such as the cross-linked polysaccharide support described hereinabove. As shown in the drawing, the aqueous ester mixture is fed to the top of the hydrolysis reaction column, but it will be understood that the direction of flow is a matter of convenience, and is not critical. The aqueous ester mixture flows through catalyst bed 7 resulting in selective hydrolysis of L-ester to L-acid and producing a product mixture which exits the bottom of the column in the configuration shown. Residence time in the column may range from less than an hour to 36 hours, but is preferably between about 4 hours and about 24 hours. Optionally, the residence time may be sufficient to achieve the desired conversion, e.g., 50%, in a single pass. However, where the conditions are highly selective, the column may be operated with a much shorter residence time to provide only partial conversion, with intact ester being recycled to the column inlet after separation from the acid component. Operation at less than 50% conversion may also be preferred where competitive chemical hydrolysis may otherwise compromise selectivity. As further described below, unhydrolyzed ester may be recycled to the inlet of the reaction column, preferably after being subjected to a racemization step.

The product mixture exiting the column is delivered to an extraction vessel 11 where it is contacted with a combination of aqueous and water-immiscible organic extractants, thereby partitioning α-hydroxy acid to the aqueous phase and unreacted ester to the organic phase. Preferably, the aqueous phase is slightly to moderately alkaline, e.g., it may comprise a dilute alkali metal bicarbonate solution, to enhance partition of the acid to that phase. After contact, the phases are allowed to settle or, preferably delivered to a liquid/solid separator such as a centrifuge 13 to promote complete and rapid separation. The aqueous phase enriched in L-α-hydroxy acid may be removed from the process as product.

Optionally, the organic phase, which is enriched in D-ester, is recycled to the inlet of the reaction column 9 for further conversion to L-acid. Although not shown in the drawing, the D-ester rich organic fraction is preferably racemized prior to recycle. If further enrichment of the acid phase is desired, it may be contacted with additional alcohol in a re-esterification reactor 15, and the resultant re-esterification product mixed with the supply of ester to form the feed to the hydrolysis reactor.

Animal Feed Supplements

Depending upon the desired application, enantiomericly enriched α-hydroxy acid mixtures of the present invention comprising an enantiomeric excess of an one α-hydroxy acid stereoisomer may be provided to animals as an amino acid supplement. Animals which may be provided enantiomerically enriched α-hydroxy acid mixtures of the present invention include humans, ruminants such as dairy cows, lactating diary cows, dairy calves, beef cattle, sheep, and goats; aquaculture such as fish and crustaceans; livestock such as swine and horses; and poultry such as chickens, turkeys, and hatchlings thereof.

The enantiomerically enriched α-hydroxy acid mixtures may also be provided to animals as an appetite suppressant.

In one embodiment, the amino acid supplement comprises an α-hydroxy acid mixture comprising an enantiomeric excess of an L-isomer of an α-hydroxy acid analog of an α-amino acid. The α-hydroxy acid analog of an α-amino acid is selected from the group consisting of the α-hydroxy acid of asparagine, alanine, valine, leucine, isoleucine, phenylalanine, proline, serine, threonine, cysteine, methionine, tryptophan, tyrosine, glutamine, aspartic acid, glutamic acid, lysine, arginine, and histidine.

In another embodiment, the amino acid supplement comprises an α-hydroxy acid mixture comprising an enantiomeric excess of an L-isomer of an α-hydroxy acid of an α-amino acid selected from the group consisting of the α-hydroxy acid of isoleucine, phenylalanine, leucine, lysine, methionine, threonine, tryptophan, histidine and valine.

In a number of highly useful embodiments, the amino acid supplement comprises an α-hydroxy acid mixture comprising an enantiomeric excess of an L-isomer of an α-hydroxy acid of an α-amino acid wherein the L-isomer is selected from the group consisting of the α-hydroxy acid of lysine and methionine. In the latter instance, the amino acid supplement comprises an α-hydroxy acid mixture comprising an enantiomeric excess of an L-isomer of 2-hydroxy-4-(methylthio)butyric acid.

Alternatively, the amino acid supplement comprises an α-hydroxy acid mixture comprising an enantiomeric excess of a D-isomer of an α-hydroxy acid of an α-amino acid. The α-hydroxy acid of an α-amino acid is selected from the group consisting of the α-hydroxy acid of asparagine, alanine, valine, leucine, isoleucine, phenylalanine, proline, serine, threonine, cysteine, methionine, tryptophan, tyrosine, glutamine, aspartic acid, glutamic acid, lysine, arginine, and histidine.

In another embodiment, amino acid supplement comprises an α-hydroxy acid mixture comprising an enantiomeric excess of an D-isomer of an α-hydroxy acid of an α-amino acid wherein the D-isomer is selected from the group consisting of the α-hydroxy acid of isoleucine, phenylalanine, leucine, lysine, methionine, threonine, tryptophan, histidine and valine.

Advantageously, the amino acid supplement may comprise an α-hydroxy acid mixture comprising an enantiomeric excess of an D-isomer of an α-hydroxy acid of an α-amino acid wherein the D-isomer is selected from the group consisting of the α-hydroxy acid of lysine or methionine.

In the latter case, the amino acid supplement comprises an α-hydroxy acid mixture comprising an enantiomeric excess of an D-isomer of 2-hydroxy-4-(methylthio)butyric acid.

The amino acid supplement mixtures comprising an enantiomeric excess of one stereoisomer of an α-hydroxy acid of an α-amino acid may be fed or otherwise administered orally, or sprayed into the eye, ear or nasal cavity of an animal, preferably a ruminant. Alternatively, the compositions may be injected, or administered bucchally (i.e., to the gums), sublingually (i.e., beneath the tongue) or rectally.

The following Examples set forth one approach that may be used to carry out the present invention. Accordingly, these Examples should not be interpreted in a limiting sense.

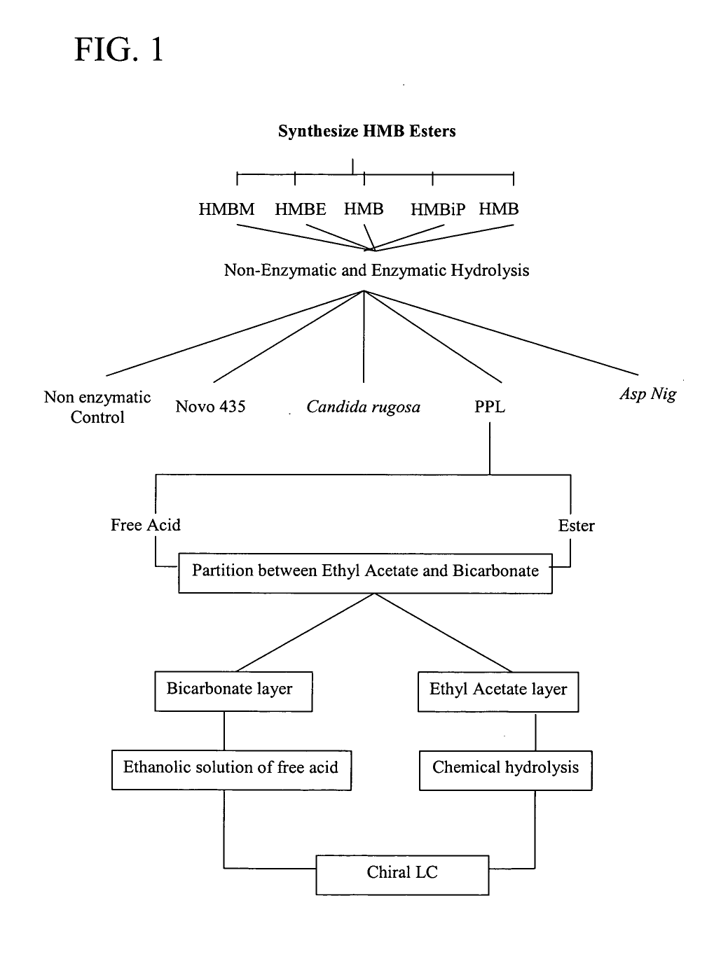

The following examples demonstrate the synthesis of an ester of an α-hydroxy acid, HMB, followed by enantioselective hydrolysis of the HMB-ester with a suitable lipase to produce enantioenriched α-hydroxy acids of HMB. Each of the Examples were carried out in accordance with the scheme depicted in FIG. 1.

### EXAMPLE 1

The following example demonstrates synthesis of HMB-methyl ester, followed by hydrolysis of HMB-methyl ester to produce an α-hydroxy acid of HMB. The hydrolysis of HMB-methyl ester was carried out both enzyme-free and with suitable lipases (Aspergillus niger, Candida rugosa, Novo 435 and porcine pancreatic lipase)as enzymes.

Preparation of HMB-ethyl ester:

Preparation of HMB-methyl ester proceeded by adding a racemic mixture of HMB (10 g) to a 200 mL round bottom flask containing methanol (40 mL). The contents of the flask were then acidified with hydrogen chloride gas for a period of 45 minutes and refluxed for a period of approximately 8 hours at 85 <C. HMB-methyl ester was recovered from the reaction mixture by removing the excess alcohol by rotary evaporation.

Determination of Yield HMB-methyl ester:

HMB-methyl ester yield was determined through liquid chromatographic analysis of the reaction product carried out with a HPLC system (Model L-7000 Hitachi High Technologies Inc., San Jose, Calif.) which included a model L-7100 piston pump (Hitachi High Technologies Inc., San Jose, Calif.) a model L-7300 column oven (Hitachi High Technologies Inc., San Jose, Calif.), a model L-7200 autosampler (Hitachi High Technologies Inc., San Jose, Calif.) and a model L-7450 UV/Vis diode array detector (Hitachi High Technologies Inc., San Jose, Calif.). Samples of the HMB-methyl ester reaction mixture were introduced into the HPLC system with a loop injection valve (Model 7400 manufactured by Rheodyne, Inc.). The analytes were separated utilizing a 250 mm×4.6 mm i.d., 5 μm C-8 reverse phase column (manufactured by P. J. Cobert Associates, Inc., St. Louis, Mo.) and the data was recorded with Hitachi HPLC software.

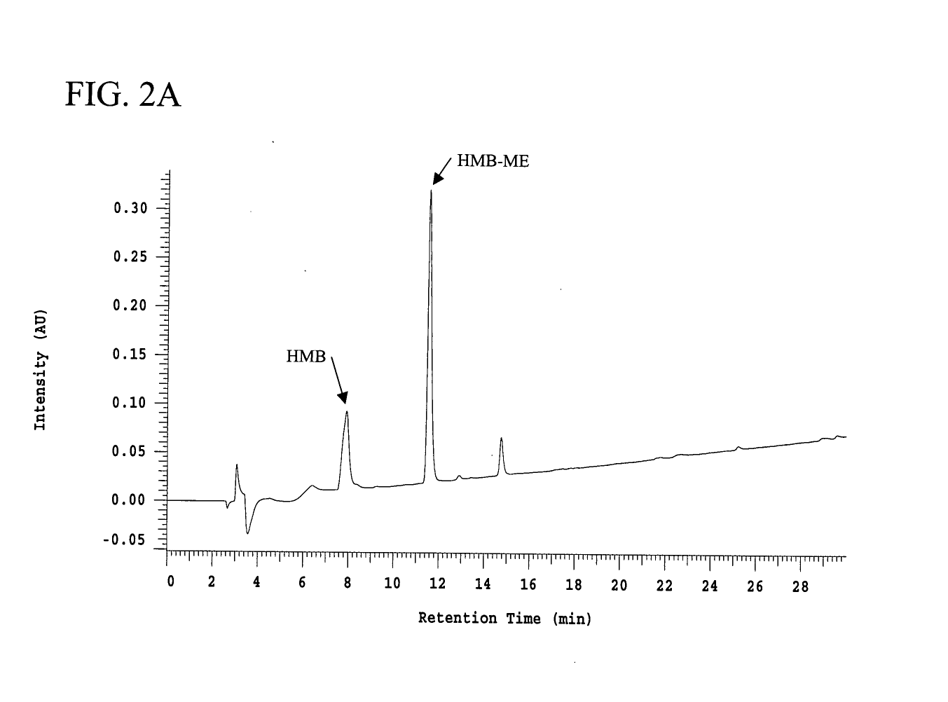

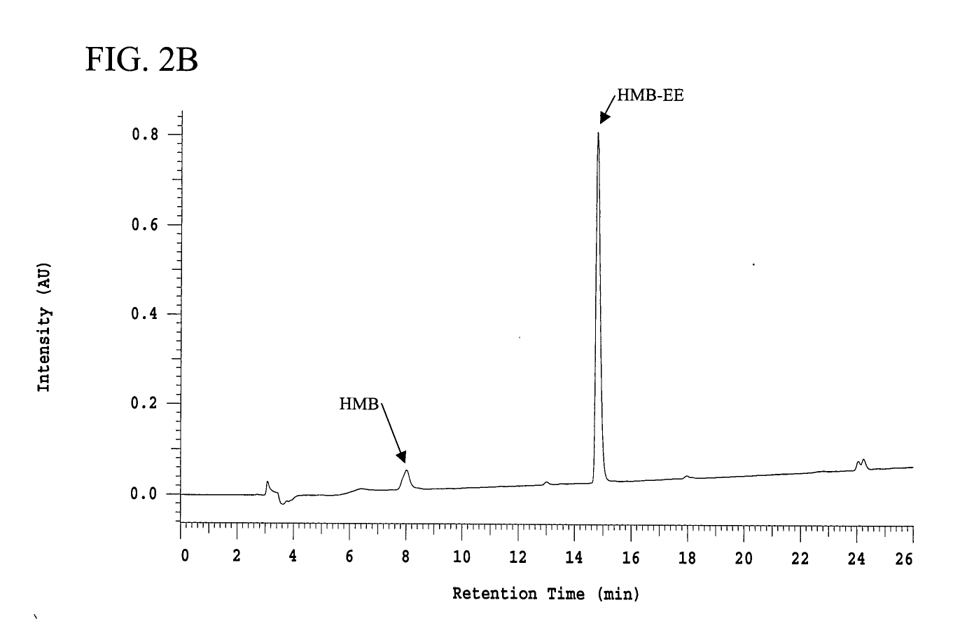

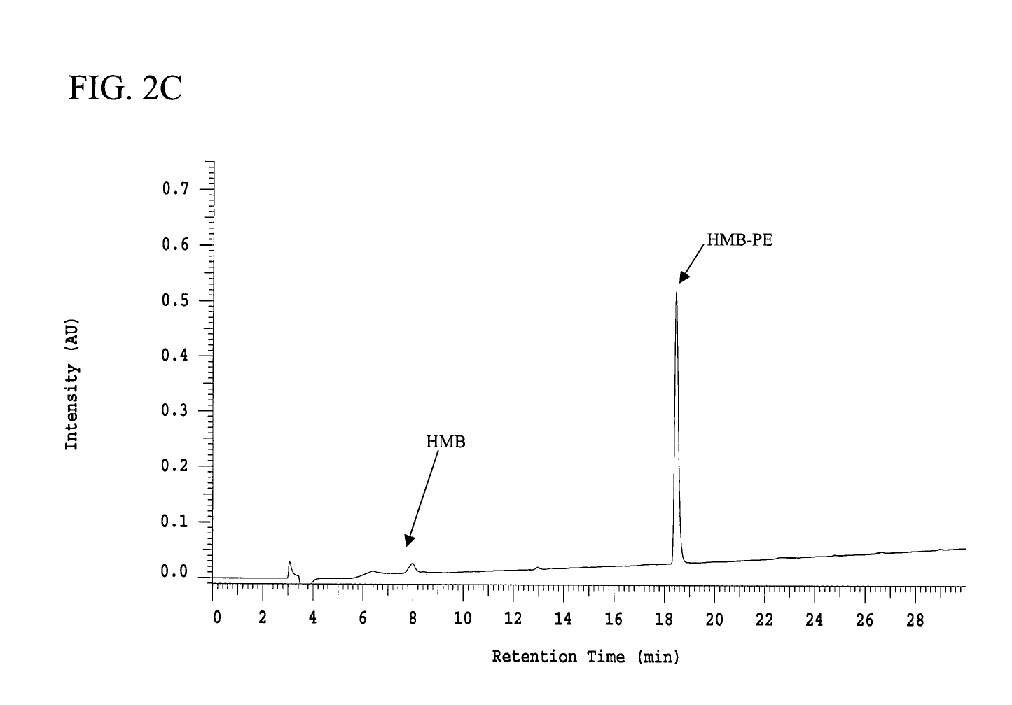

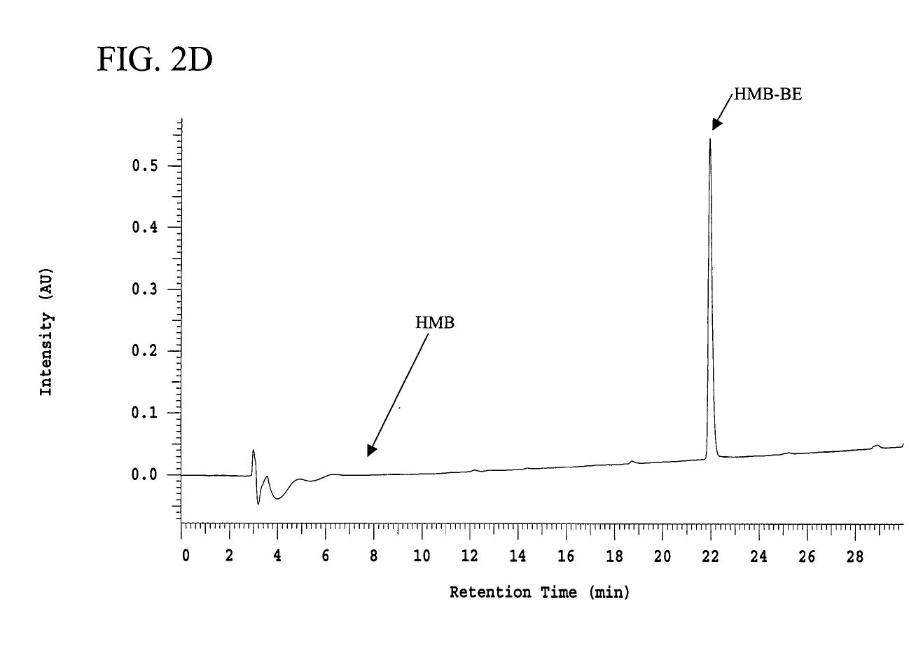

The HMB free acid and HMB-methyl ester separations were achieved with a linear gradient program in which the mobile phase composition was changed from 100% eluent A (water with 0.1% trifluoroacetic acid (TFA) to 55% eluent A and 45% eluent B (0.1% TFA in acetonitrile) over the course of a 30 minute period during which the mobile phase flow rate was maintained at 1 mL/min. The absorbance of the separated analytes was monitored at 220 nm. The chromatographic separation of HMB and HMB-methyl ester is illustrated FIG. 2A.

Non-Enzymatic Hydrolysis:

To carry out the hydrolysis of the ester, HMB-methyl ester (1 mM) was added to a 40 mL reaction vial along with water (12 mL) and a 0.1 M phosphate buffer having a pH of 7.2 (2 mL). The reaction was carried out at a constant temperature of, approximately 25° C. and constant stirring over a period of 24 hours. During the course of the reaction, the pH of the reaction mixture was maintained at 7.2. After the reaction was complete, the reaction solvents were removed from the reaction mixture with a rotary evaporator.

Lipase Mediated Hydrolysis:

Lipase mediated hydrolysis of HMB-methyl ester to produce an α-hydroxy acid of HMB was carried out on four separate occasions utilizing different lipases. These lipases were: Aspergillus niger, Candida rugosa, Novo 438 (Candida antarctica), and porcine pancreatic lipase (PPL).

In one lipase mediated hydrolysis, HMB-methyl ester (1 mM) was added to a 40 mL reaction vial along with water (12 mL), a 0.1 M phosphate buffer with a pH of 7.2 (2 mL) and Aspergillus niger (120 mg).

In another lipase mediated hydrolysis, HMB-methyl ester (1 mM) was added to a 40 mL reaction vial along with water (12 mL), a 0.1 M phosphate buffer with a pH of 7.2 (2 mL) and Candida rugosa (120 mg).

In another lipase mediated hydrolysis, HMB-methyl ester (1 mM) was added to a 40 mL reaction vial along with water (12 mL), a 0.1 M phosphate buffer with a pH of 7.2 (2 mL) which was contacted with an immobilized sample of Novo 438 (Candida antarctica) (6 mg).

In another lipase mediated hydrolysis, HMB-methyl ester (1 mM) was added to a 40 mL reaction vial along with water (12 mL), a 0.1 M phosphate buffer with a pH of 7.2 (2 mL) and PPL (120 mg).

The non-enzymatic hydrolysis and the lipase mediated hydrolysis reactions were each carried out at a constant temperature of approximately 25° C. with constant stirring over the course of a 24 hour period. During the course of reaction pH of the reaction mixture was maintained at 7.2. After the reaction was complete, the reaction solvents were removed from the reaction mixture with a rotary evaporator.

Product Recovery

After each of the hydrolysis reactions, the dry hydrolysis reaction product containing the α-hydroxy acid of HMB was dissolved in ethanol (10 mL), a sample of the reaction product solution (1 mL) was taken and diluted with additional ethanol (9 mL) and the further diluted solution was filtered through a filter having a pore size of 2 μm (Model manufactured by Millipore Corp. of Bedford, Mass.). The filtered extract was then introduced into the liquid chromatograph and the free acid and residual ester were separated on a C-8 reverse phase liquid chromatography column of the type described above.

Ethanol was removed from the remaining product mixture by rotary evaporation and the α-hydroxy acid product was re-dissolved in ethyl acetate (5 mL). The solution containing the α-hydroxy acid product was transferred to a 15 mL centrifuge tube to which 1 M sodium bicarbonate (6 mL) was added after which the centrifuge tube was shaken for a period of about five minutes, thus forming an ethyl acetate layer containing HMB-methyl ester and a sodium bicarbonate layer containing D- and L-enantiomers of the α-hydroxy acid, HMB. These layers were then separated with a centrifuge and each of the separated layers were brought to near dryness with a rotary evaporator and the residue from the bicarbonate layer was dissolved in ethanol (5 mL) to form a solution which was filtered and analyzed for D- and L-enantiomers of HMB by chiral liquid chromatography in accordance with the method set forth below.

The dry residue from the ethyl acetate layer containing HMB-methyl ester was hydrolyzed with 6N HCl (2 mL) for a period of two hours to produce HMB which was solubilized in ethanol. The ethanol solutions were filtered and analyzed for D- and L-enantiomers of HMB by chiral liquid chromatography in accordance with the method set forth below.

Separation of the D,L Forms of the Racemic Mixture of the α-hydroxy Acid of HMB:

Separation of the D- and L-forms of the α-hydroxy acid of HMB present in the sodium bicarbonate phase was carried out by liquid chromatography (LC) with a UV absorbance detector in tandem with electrospray ionization mass spectrometry in the negative mode (ESI(−)MS). The LC-UV-ESI(−)MS separation was achieved with an isocratic elution. The electrospray ionization mass spectrometer utilized was manufactured by Hitachi High Technologies (Model M-8000). The mobile phase composition consisted of 70% ammonium acetate buffer (pH 4, 30 mM) and 30% methanol. A macrocyclic antibiotic based column (Chirobiotic-TAG), manufactured by Advanced Separation Technologies, Inc., Whippany, N.J., was used for the chiral liquid chromatography.

The following Table shows the ratio of α-hydroxy acid (HMB) produced to HMB-methyl ester hydrolyzed for the non-enzymatic catalyzed hydrolysis and each of the lipase catalyzed hydrolysis reactions HMB-methyl ester.

TABLE I

| | |
|---|---|
| HMB/HMB-methyl ester ratio for non-enzyme | 1.45 |
| catalyzed hydrolysis | |
| HMB/HMB-methyl ester ratio for Aspergillus | 4.55 |
| niger lipase catalyzed hydrolysis | |
| HMB/HMB-methyl ester ratio for Candida | 62.37 |
| rugosa lipase catalyzed hydrolysis | |
| HMB/HMB-methyl ester ratio for Novo 435 | 128 |
| (Candida antarctica) lipase catalyzed | |
| hydrolysis | |
| HMB/HMB-methyl ester ratio for PPL, lipase | 2.53 |
| catalyzed hydrolysis | |

### EXAMPLE 2

The following Example was carried out in accordance with the protocol set forth above in Example 1 utilizing HMB-ethyl ester.

The following Table shows the ratio of α-hydroxy acid (HMB) produced to HMB-ethyl ester hydrolyzed for the non-enzymatic catalyzed hydrolysis and each of the lipase catalyzed hydrolysis reactions HMB-ethyl ester.

TABLE II

| | |
|---|---|
| HMB/HMB-ethyl ester ratio for non-enzyme | 0.53 |
| catalyzed hydrolysis | |
| HMB/HMB-ethyl ester ratio for Aspergillus | 16.71 |
| niger lipase catalyzed hydrolysis | |
| HMB/HMB-ethyl ester ratio for Candida rugosa | 7.84 |
| lipase catalyzed hydrolysis | |
| HMB/HMB-ethyl ester ratio for Novo 435 | — |
| (Candida antarctica) lipase catalyzed | |
| hydrolysis | |
| HMB/HMB-ethyl ester ratio for PPL lipase | 0.94 |
| catalyzed hydrolysis | |

### EXAMPLE 3

The following Example was carried out in accordance with the protocol set forth above in Example 1 utilizing HMB-propyl ester.

The following Table shows the ratio of α-hydroxy acid (HMB) produced to HMB-propyl ester hydrolyzed for the non-enzymatic catalyzed hydrolysis and each of the lipase catalyzed hydrolysis reactions HMB-propyl ester.

TABLE III

| | |
|---|---|
| HMB/HMB-propyl ester ratio for non-enzyme | 0.09 |
| catalyzed hydrolysis | |
| HMB/HMB-propyl ester ratio for Aspergillus | 149.24 |
| niger lipase catalyzed hydrolysis | |
| HMB/HMB-propyl ester ratio for Candida | 84.45 |
| rugosa lipase catalyzed hydrolysis | |
| HMB/HMB-propyl ester ratio for Novo 435 | 52.27 |
| (Candida antarctica) lipase catalyzed | |
| hydrolysis | |
| HMB/HMB-propyl ester ratio for PPL lipase | 1.69 |
| catalyzed hydrolysis | |

### EXAMPLE 4

The following Example was carried out in accordance with the protocol set forth above in Example 1 utilizing HMB-isopropyl ester.

The following Table shows the ratio of α-hydroxy acid (HMB) produced to HMB-isopropyl ester hydrolyzed for the non-enzymatic catalyzed hydrolysis and each of the lipase catalyzed hydrolysis reactions HMB-isopropyl ester.

TABLE IV

| | |
|---|---|
| HMB/HMB-isopropyl ester ratio for non-enzyme | 0.36 |
| catalyzed hydrolysis | |
| HMB/HMB-isopropyl ester ratio for | 0.81 |
| Aspergillus niger lipase catalyzed | |
| hydrolysis | |
| HMB/HMB-isopropyl ester ratio for Candida | 9.8 |
| rugosa lipase catalyzed hydrolysis | |
| HMB/HMB-isopropyl ester ratio for Novo 435 | 165 |
| (Candida antarctica) lipase catalyzed | |
| hydrolysis | |
| HMB/HMB-isopropyl ester ratio for PPL lipase | 3.3 |
| catalyzed hydrolysis | |

### EXAMPLE 5

The following Example was carried out in accordance with the protocol set forth above in Example 1 utilizing HMB-butyl ester.

The following Table shows the ratio of α-hydroxy acid (HMB) produced to HMB-butyl ester hydrolyzed for the non-enzymatic catalyzed hydrolysis and each of the lipase catalyzed hydrolysis reactions HMB-butyl ester.

TABLE V

| | |
|---|---|
| HMB/HMB-butyl ester ratio for non-enzyme | 0.09 |
| catalyzed hydrolysis | |
| HMB/HMB-butyl ester ratio for Aspergillus | 160.57 |
| niger lipase catalyzed hydrolysis | |
| HMB/HMB-butyl ester ratio for Candida rugosa | 42.6 |
| lipase catalyzed hydrolysis | |
| HMB/HMB-butyl ester ratio for Novo 435 | 176.3 |
| (Candida antarctica) lipase catalyzed | |
| hydrolysis | |
| HMB/HMB-butyl ester ratio for PPL lipase | 2.0 |
| catalyzed hydrolysis | |

Stereo Selectivity of the Lipase Catalyzed HMB Esters Hydrolysis

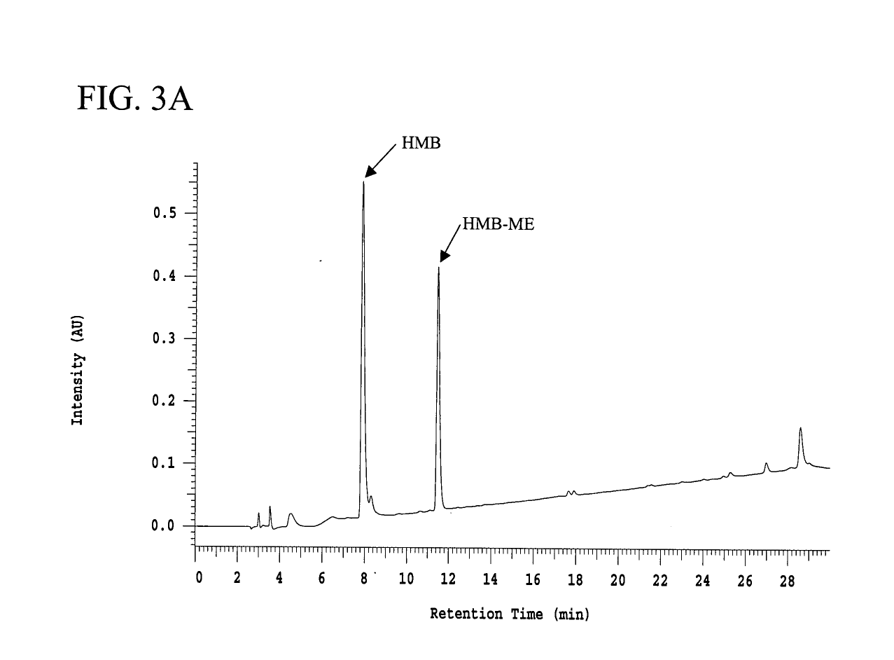

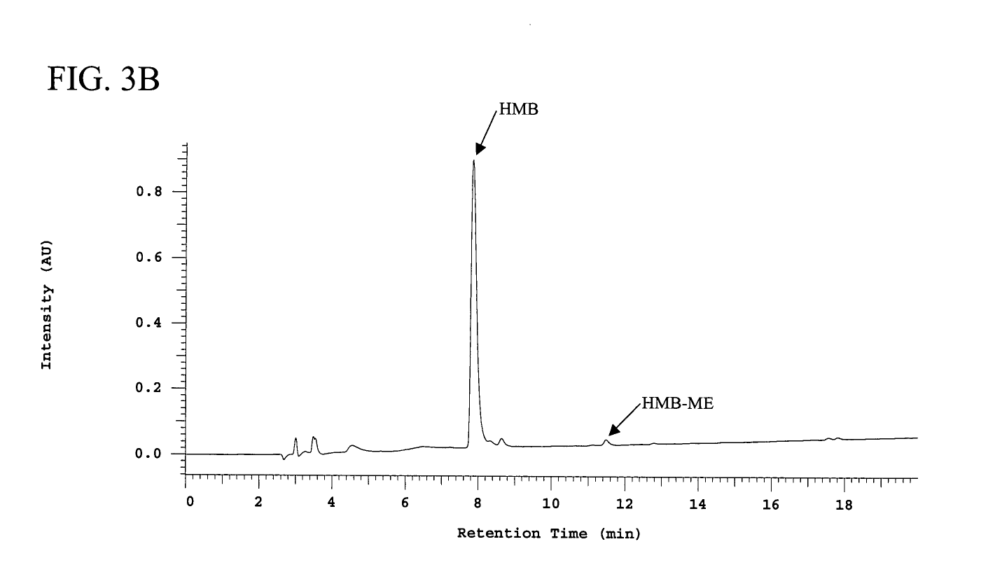

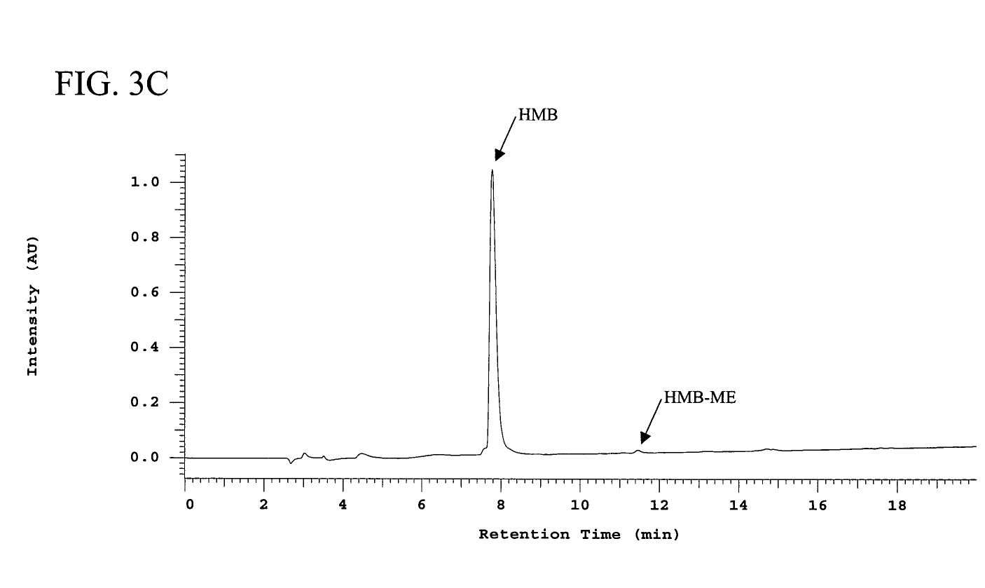

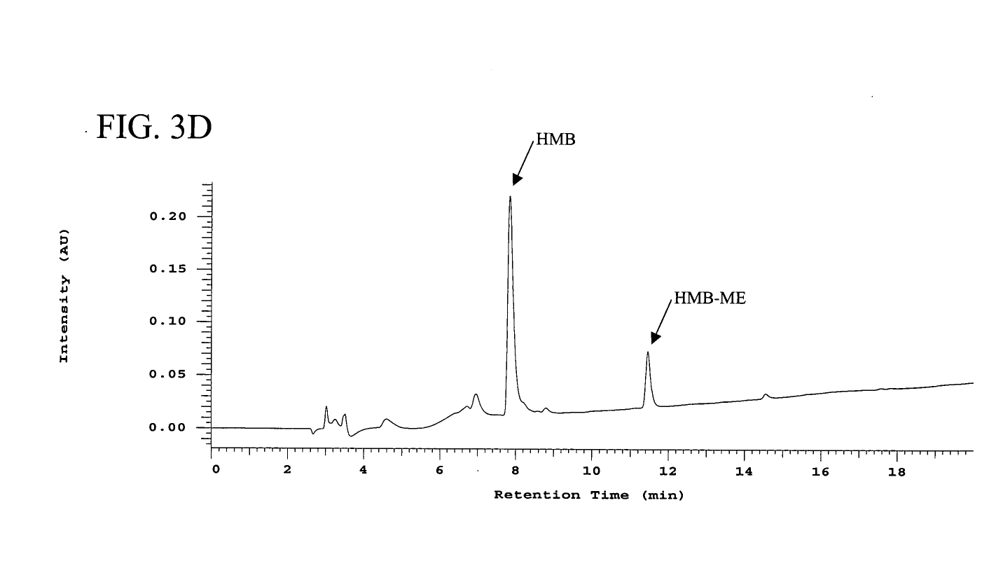

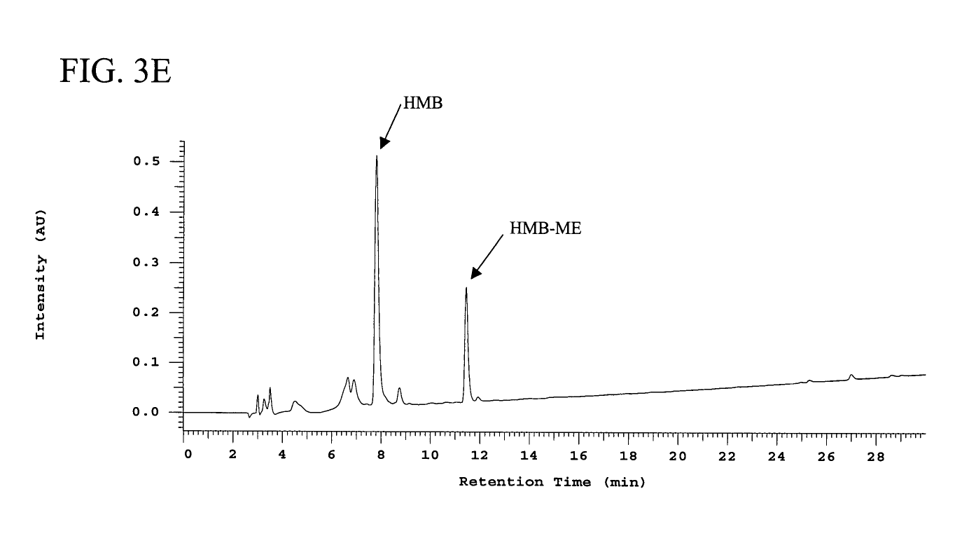

The reverse phase liquid chromatography separations of hydrolysates obtained after enzymatic hydrolysis of HMB-methyl ester are shown in FIGS. 3B-3E. The data shows that two of the lipases (Candida rugosa

, FIG. 3B, and Novo 435 FIG. 3C) catalyze HMB-methyl ester hydrolysis to near completion. The hydrolysis of HMB-methyl ester catalyzed by lipase (Aspergillus niger) and porcine pancreatic lipase (PPL) was partial indicating some enantioselectivity.

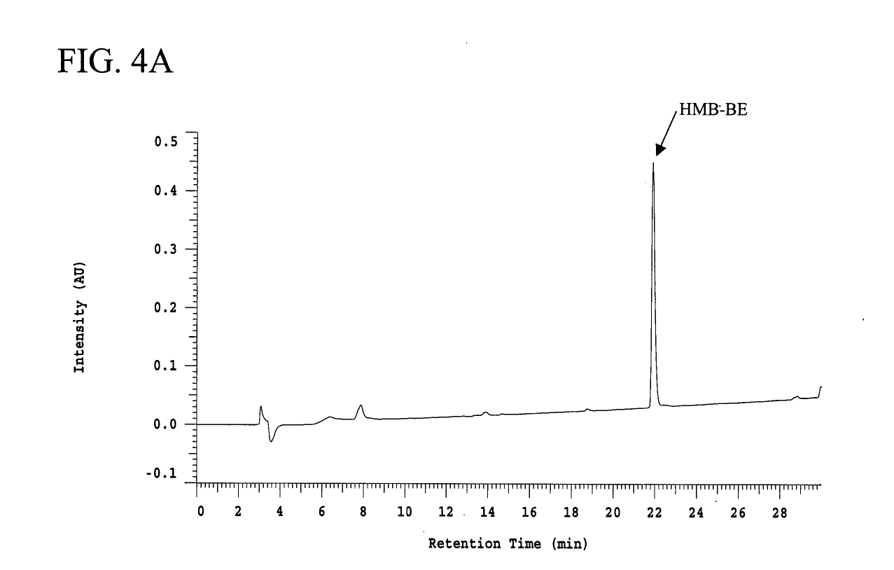

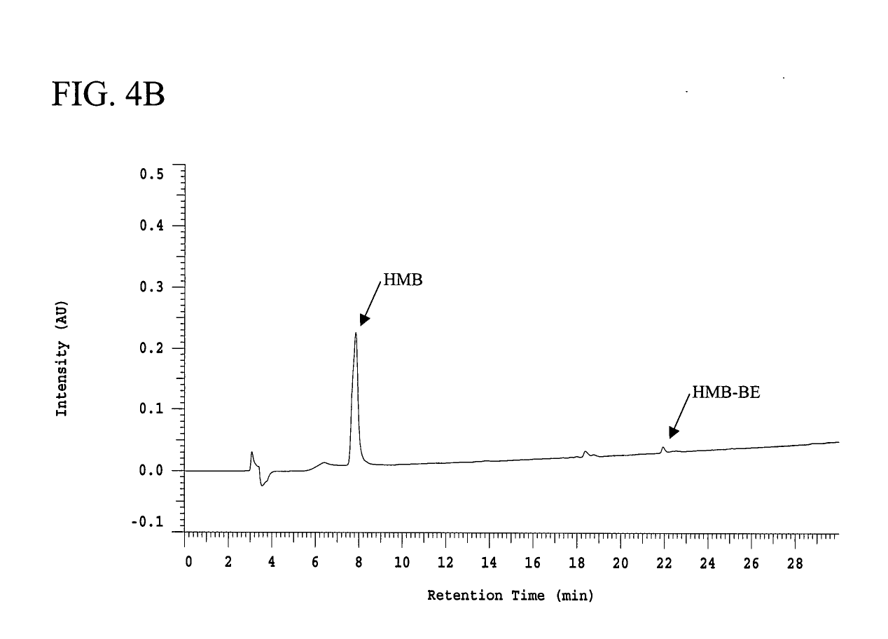

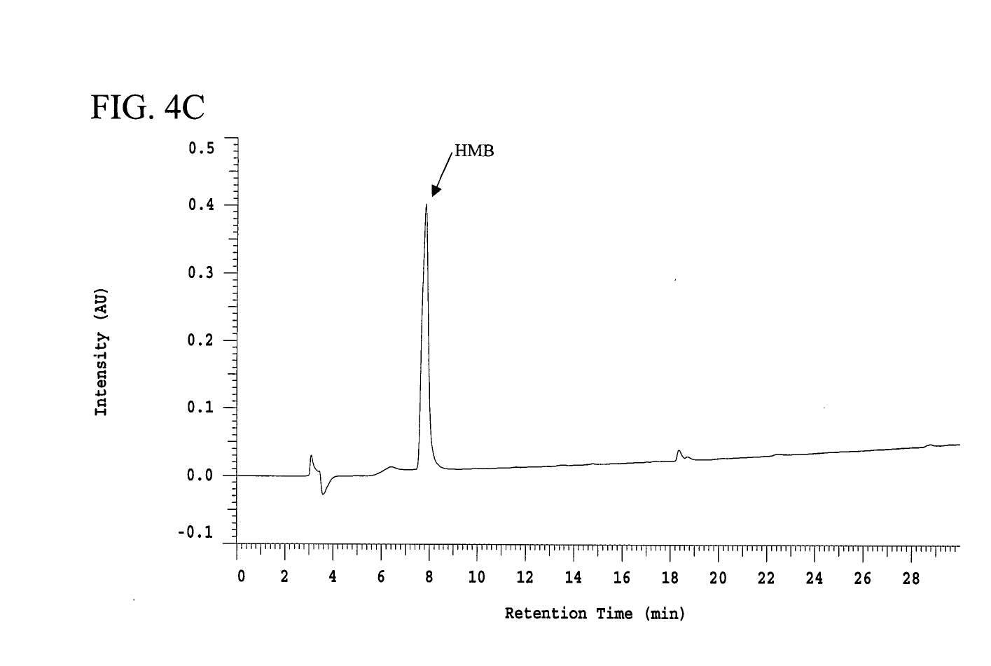

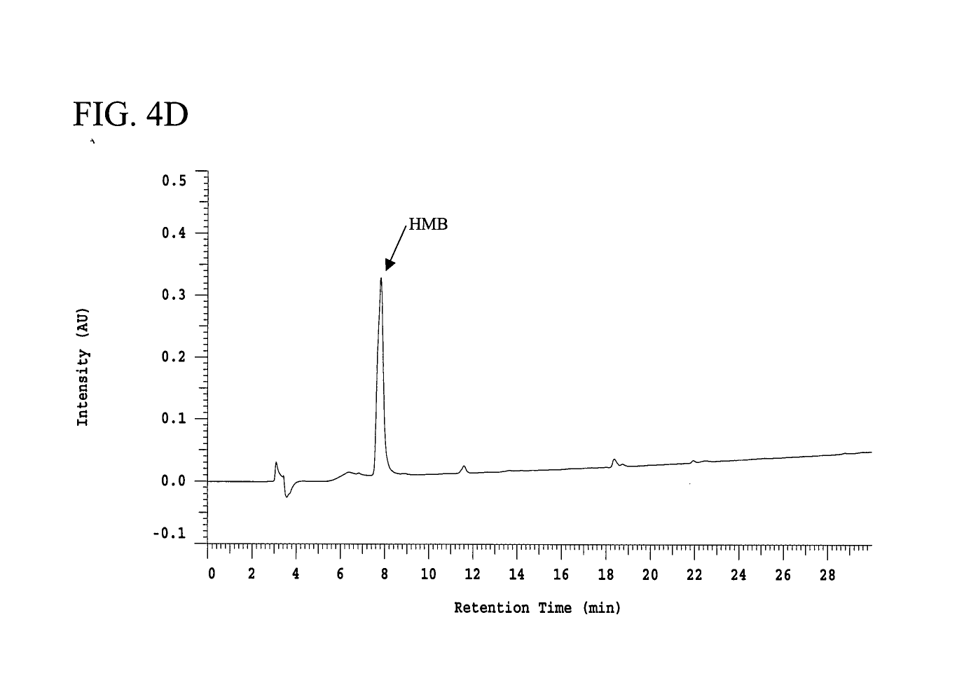

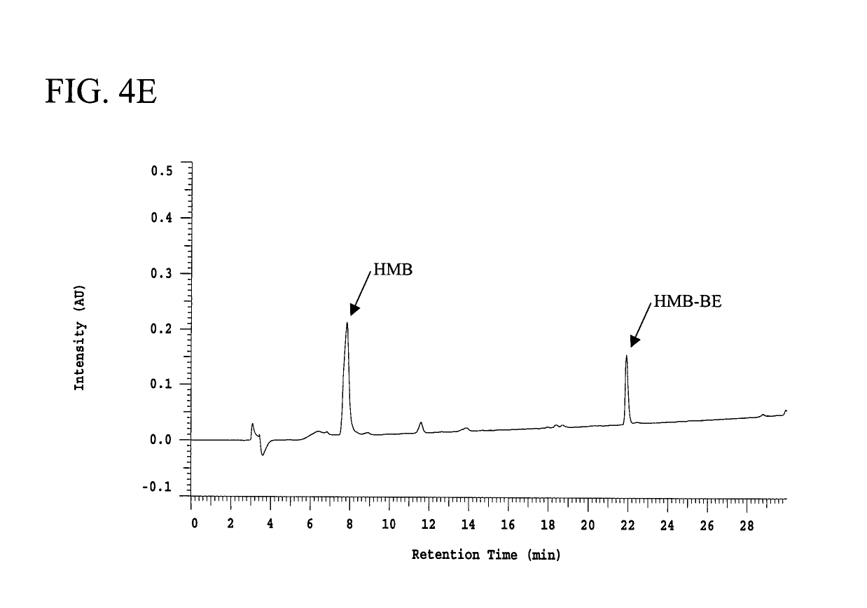

A higher degree of potential enantioselectivity was observed in the case of lipase-catalyzed hydrolysis of HMB-propyl ester and HMB-butyl ester. Chromatographic results of non-enzymatic and enzymatic HMB-butyl ester hydrolysis are shown in FIGS. 4A-4E. Results clearly indicate that HMB-butyl ester is resistant to non-enzymatic hydrolysis since less than 1% of the ester was hydrolyzed.

Essentially all of the HMB-butyl ester was hydrolyzed in the presence of lipases from Candida rugosa (FIG. 4B), Novo 435 (FIG. 4C), and Aspergillus niger (FIG. 4D) indicating little enantioselectivity, since for complete hydrolysis to occur both enantiomers need to be hydrolyzed. However, hydrolysis with PPL (FIG. 4D) was only partial indicating potential enantioselectivity.

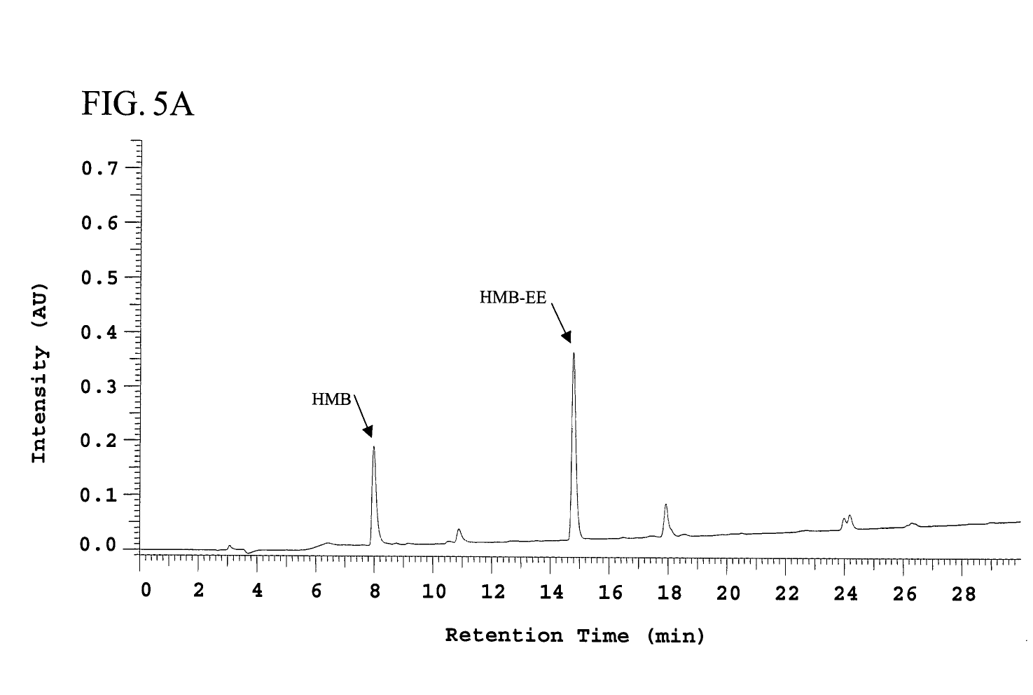

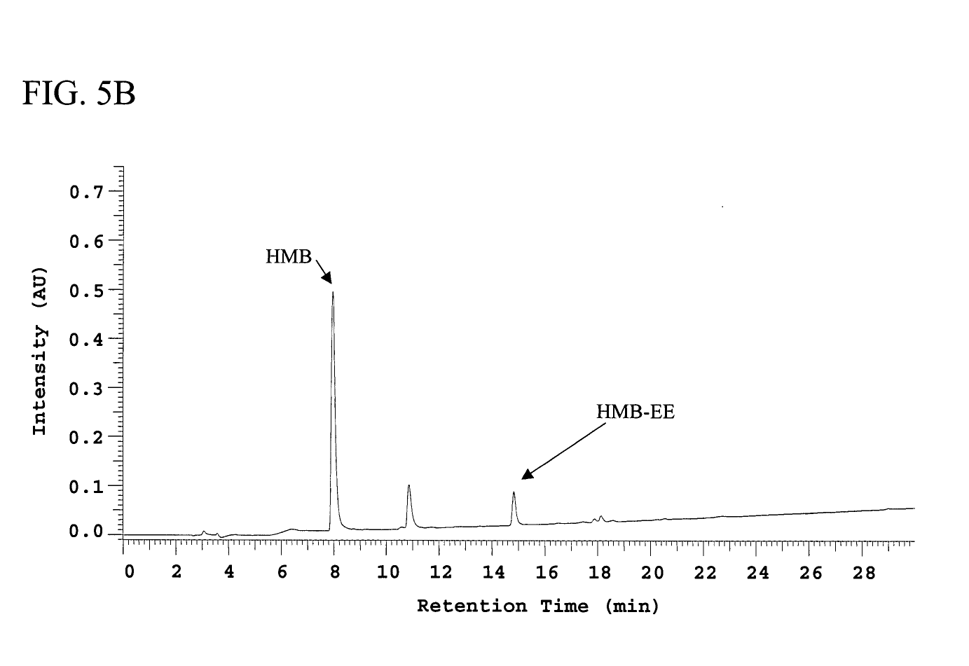

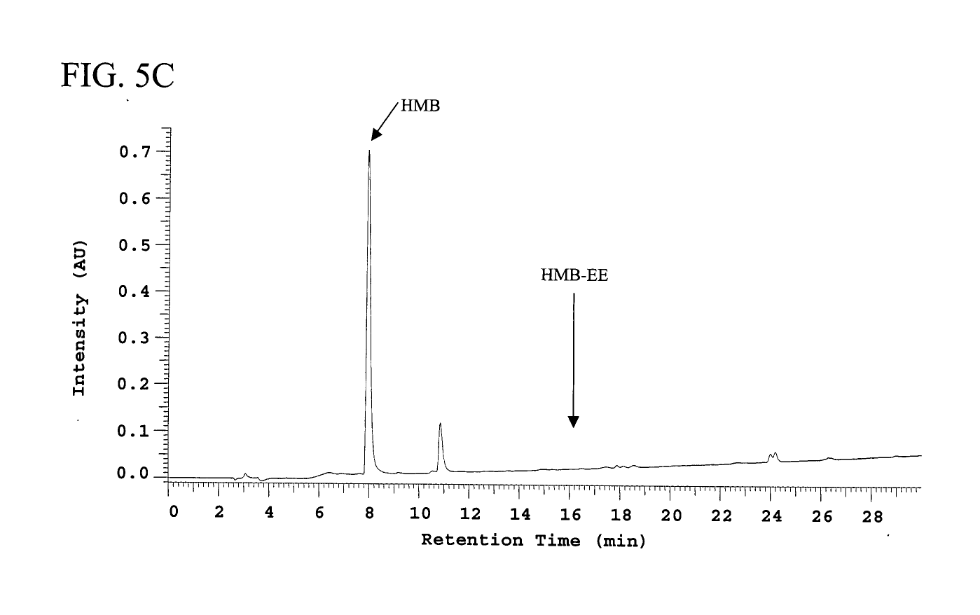

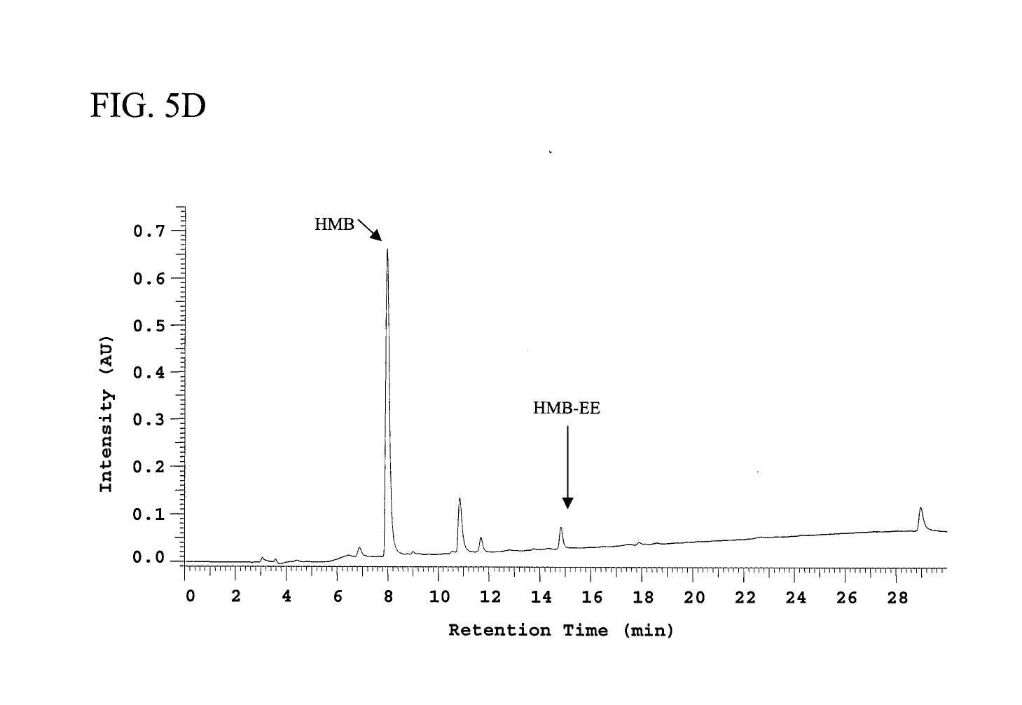

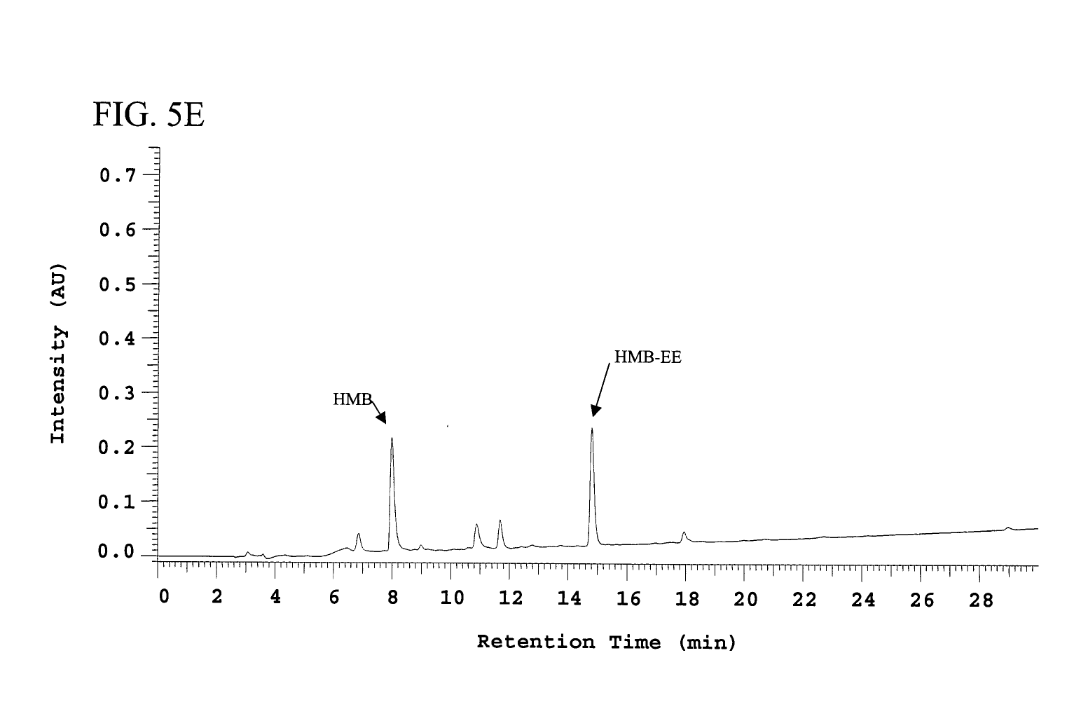

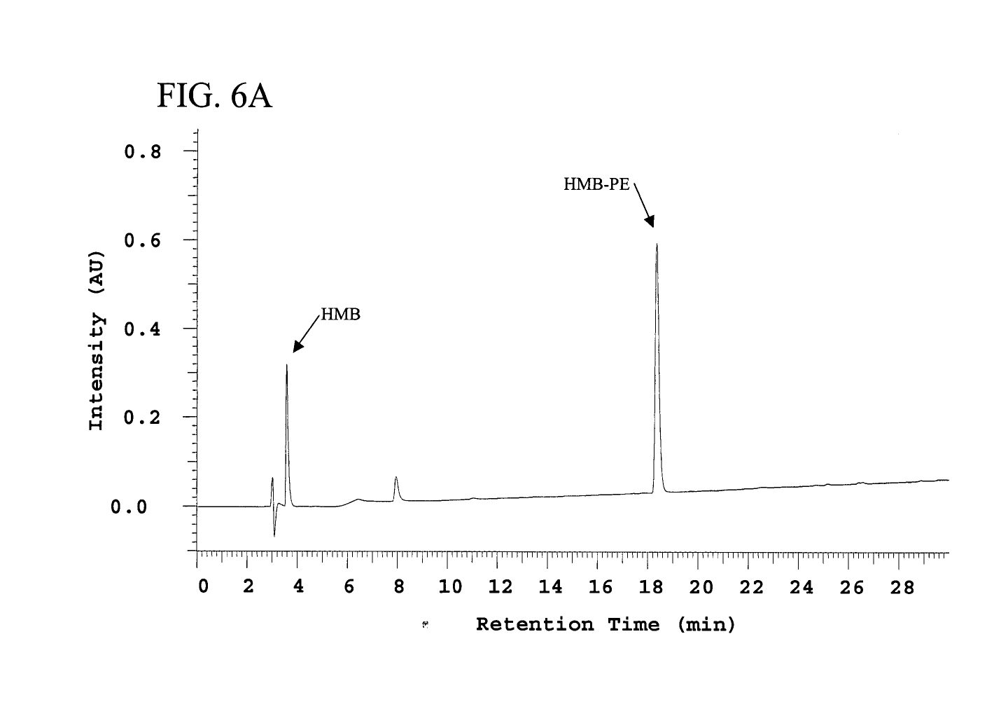

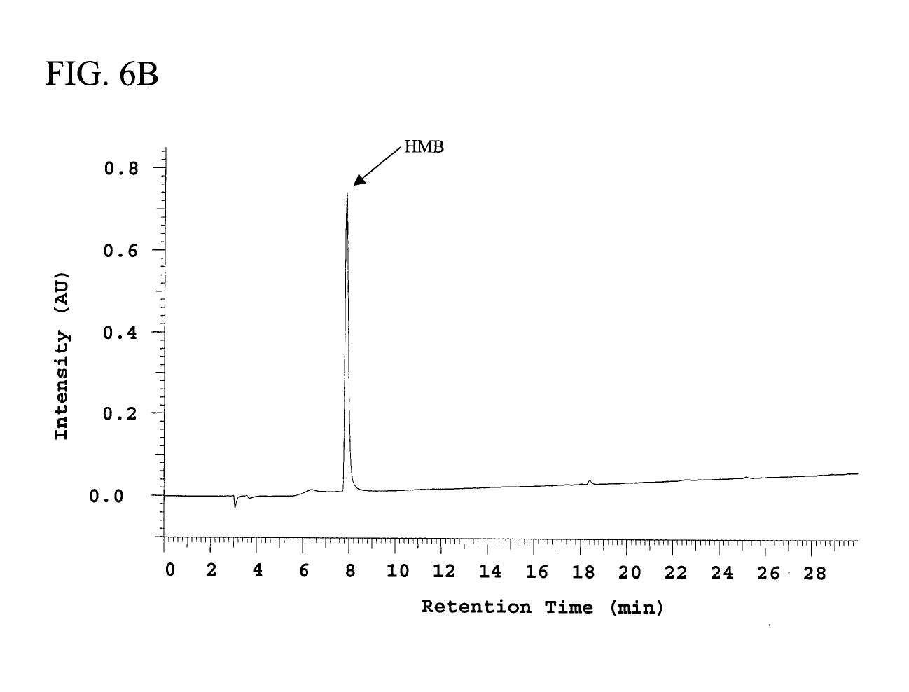

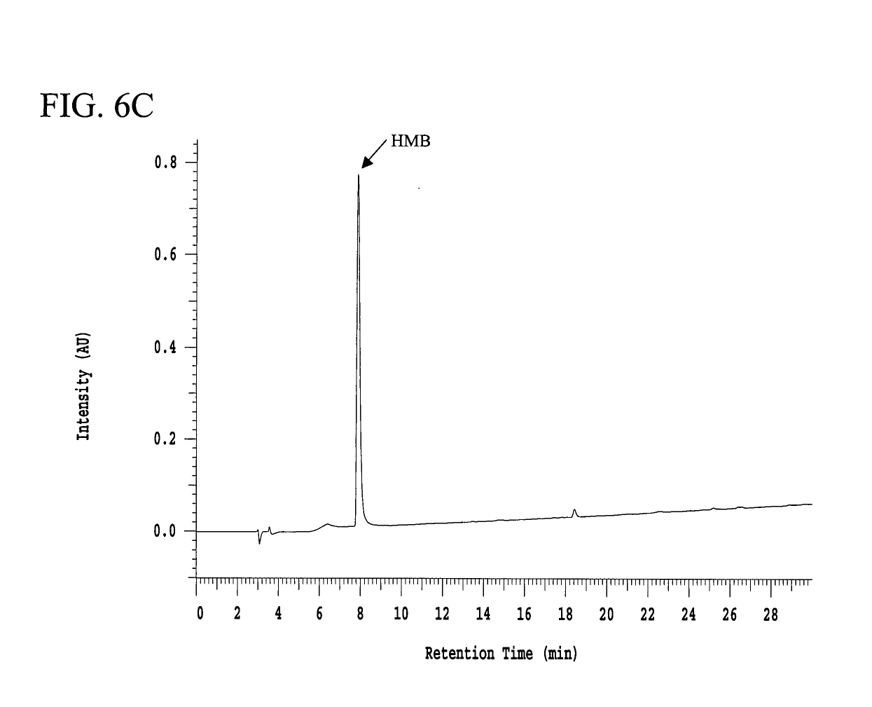

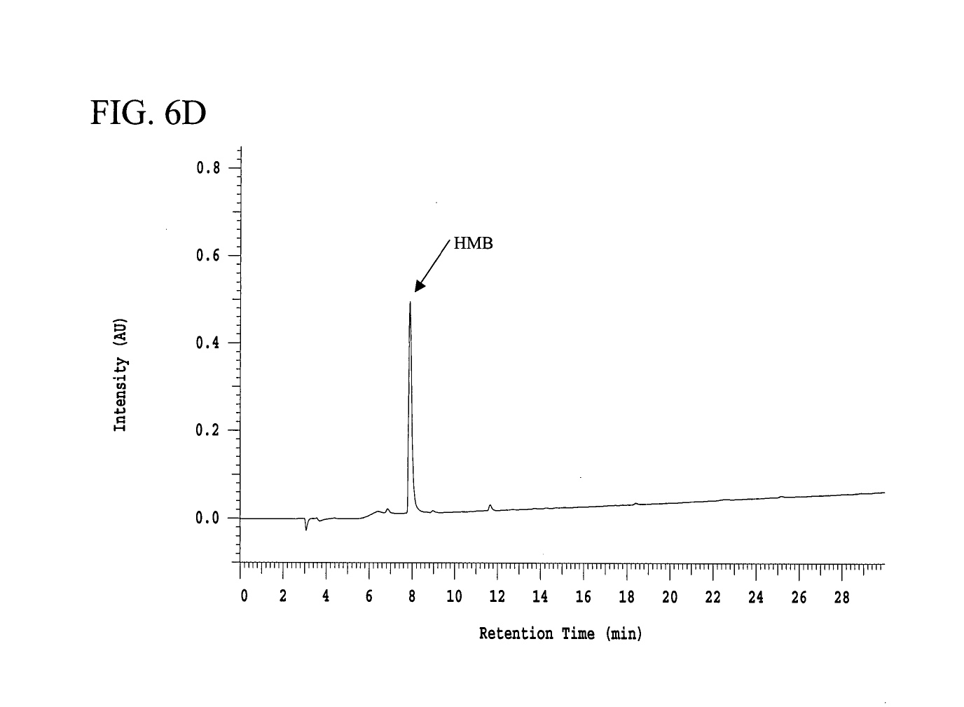

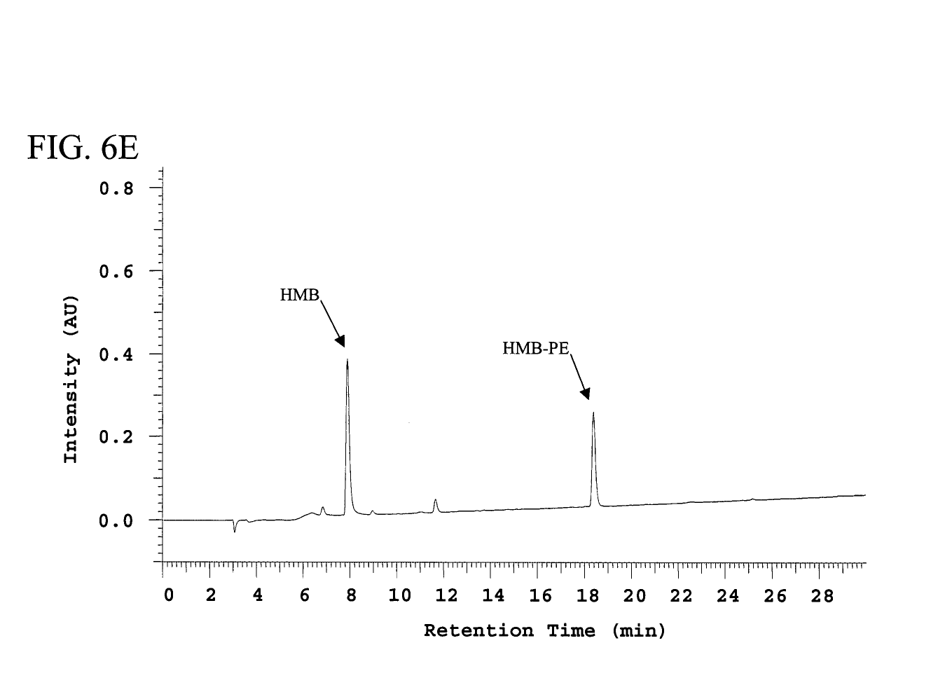

Experiments were also conducted using the ethyl and propyl esters of HMB to determine potential enantioselectivity of Candida rugosa (FIGS. 5B and 6B), Novo 435 (FIGS. 5C and 6C), Aspergillus niger (FIGS. 5D and 6D), and procine pancreatic lipase (FIGS. 5E and 6E).

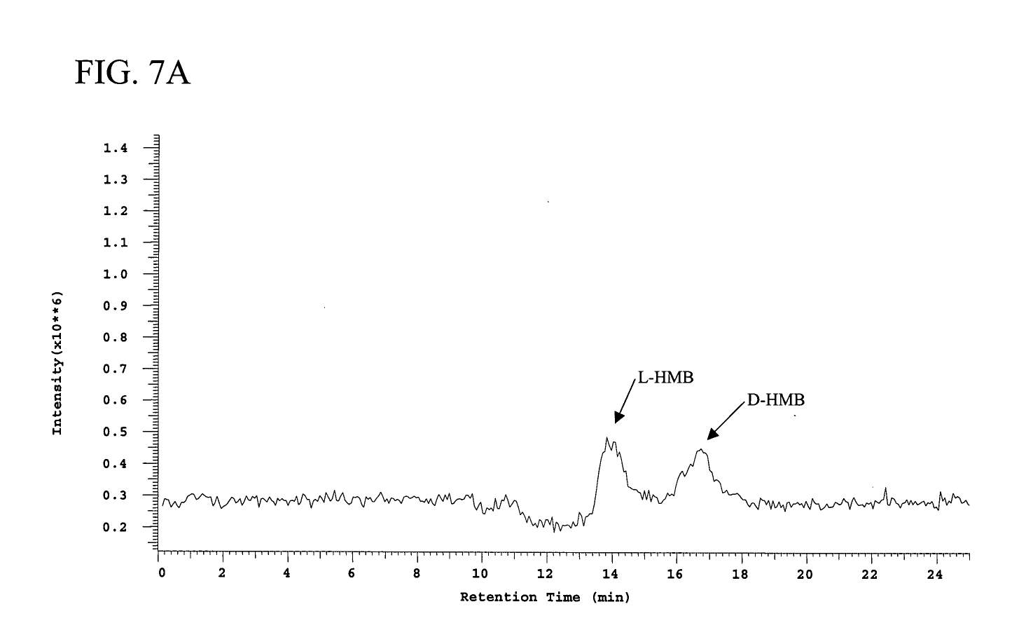

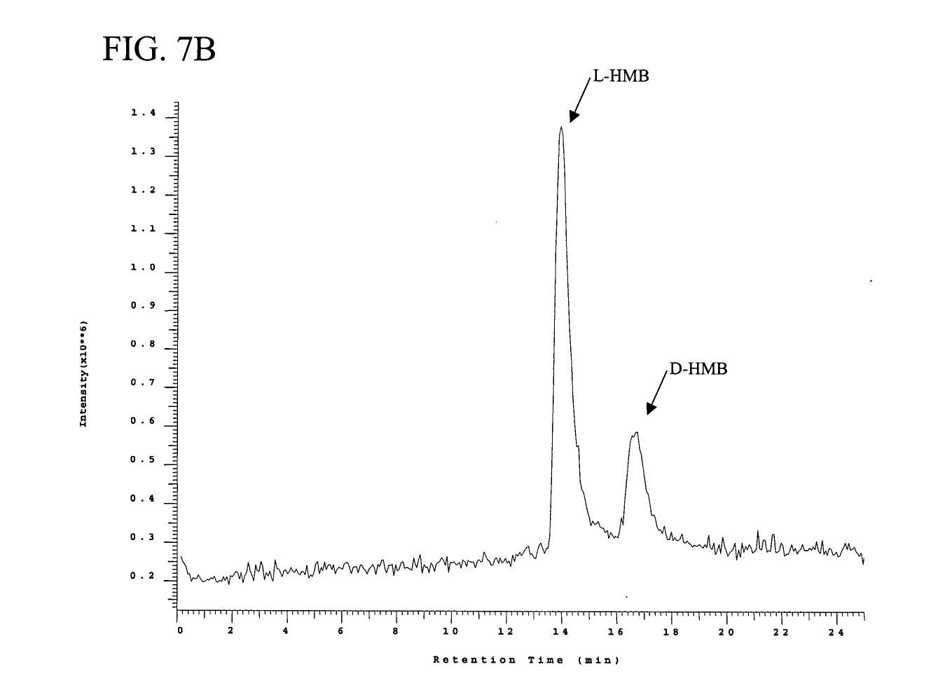

Based upon the these results, PPL catalyzed hydrolysis HMB-butyl ester was investigated further. As part of these investigations, HMB-butyl ester and HMB obtained from the hydrolysis were recovered from the reaction mixture through portioning in ethyl acetate and 0.1 mM bicarbonate solution. The HMB acid was recovered from the bicarbonate solution; it was introduced into an enantioselective HPLC system. The recovered HMB-butyl ester was hydrolyzed with 6N HCl (chemically) and then introduced into the enantioselective HPLC system. Chromatograms of HMB enantiomers obtained after non-enzymatic and PPL catalyzed HMB-butyl ester hydrolysis are illustrated in FIGS. 7A-B respectively.

The chromatogram of HMB obtained through chemical hydrolysis of HMB-butyl ester (FIG. 7A) contained both D- and L-HMB enantiomers at equal concentrations, attesting to the non-enantioselective nature of chemical hydrolysis. By contrast, the chromatogram in FIG. 7B illustrates the predominance of the L-enantiomer since its concentration was nearly four times the concentration of the D-enantiomer. Similar results were obtained from PPL catalyzed hydrolysis of HMB-propyl ester.

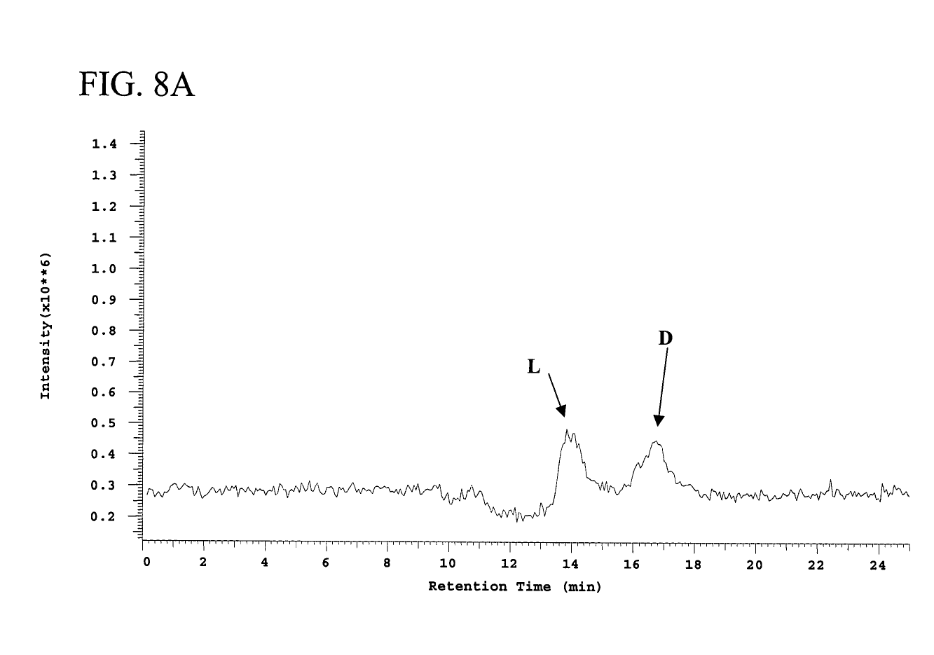

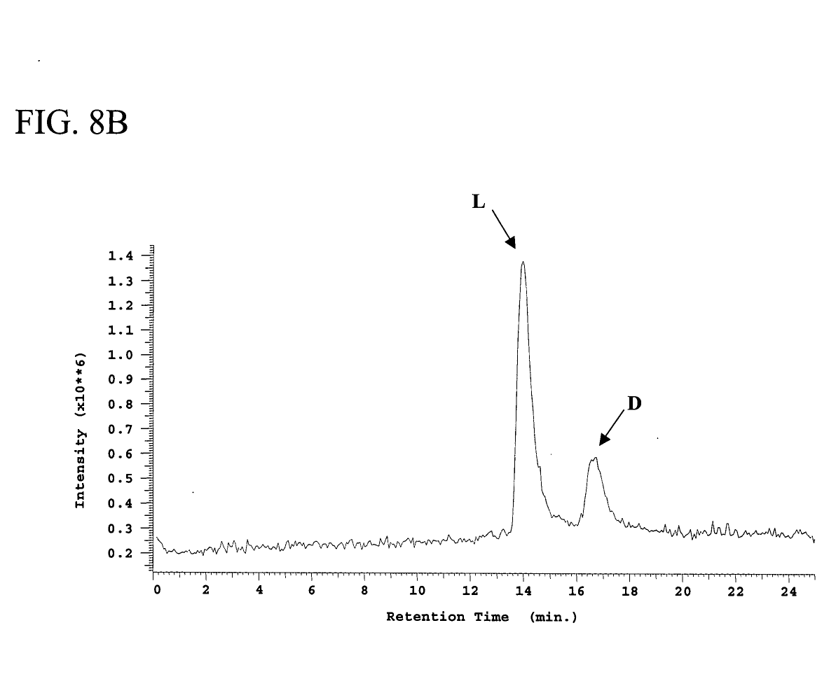

Non-enzymatic and PPL catalyzed hydrolysis of HMB-propyl ester were also investigated. Chromatograms of HMB enantiomers obtained after non-enzymatic and PPL catalyzed HMB-propyl ester hydrolysis are illustrated in FIGS. 8A-B, respectively.

Results of enzymatic hydrolysis of different HMB-esters are summarized in Table VI.

TABLE VI
Enantioselectivity of HMB-Esters Hydrolysis with Different
Lipases

| | | Free Acid | Ester |
|---|---|---|---|
| Lipase | HMB - ester | (L/D) | (L/D) |
| PPL | HMB-butyl ester | 81/19 | 19/81 |
| PPL | HMB-propyl ester | 75/25 | 22/78 |
| PPL | HMB-ethyl ester | 57/43 | 43/57 |
| Candida | HMB-ethyl ester | 62/38 | 38/62 |
| rugosa | | | |
| Aspergillus | HMB-isopropyl | 68/32 | 32/68 |
| niger | ester | | |

The highest enantioselectivity was observed during hydrolysis of HMB-propyl ester and HMB-butyl ester with the porcine pancreatic lipase (PPL). Enantioselectivity was also observed during hydrolysis of HMB-ethyl ester and HMB-isopropyl ester with lipase from Candida rugosa and Aspergillus niger respectively.

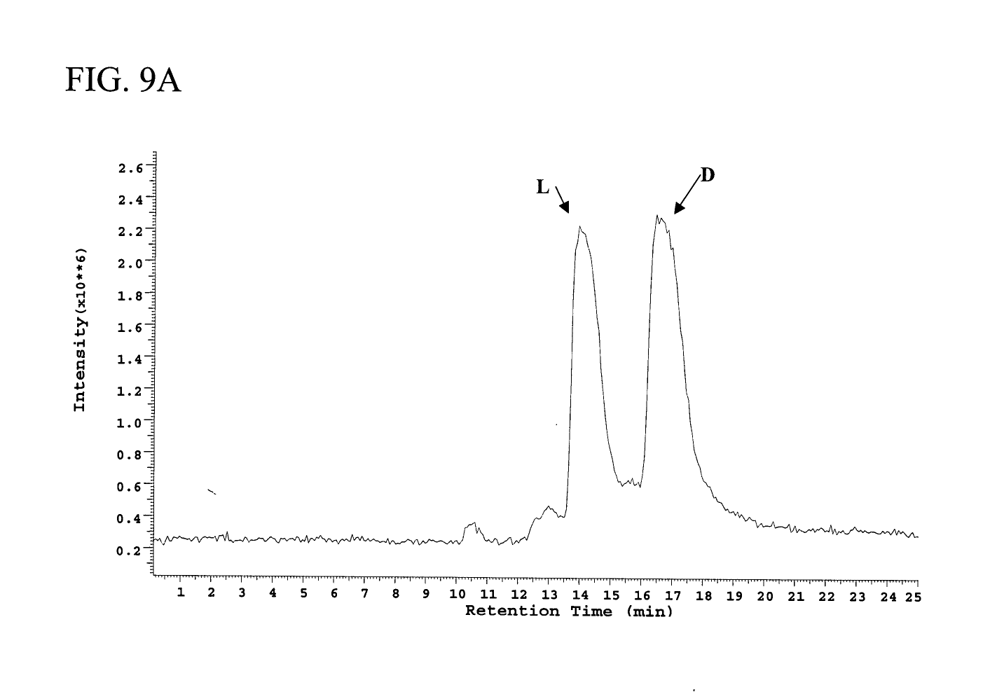

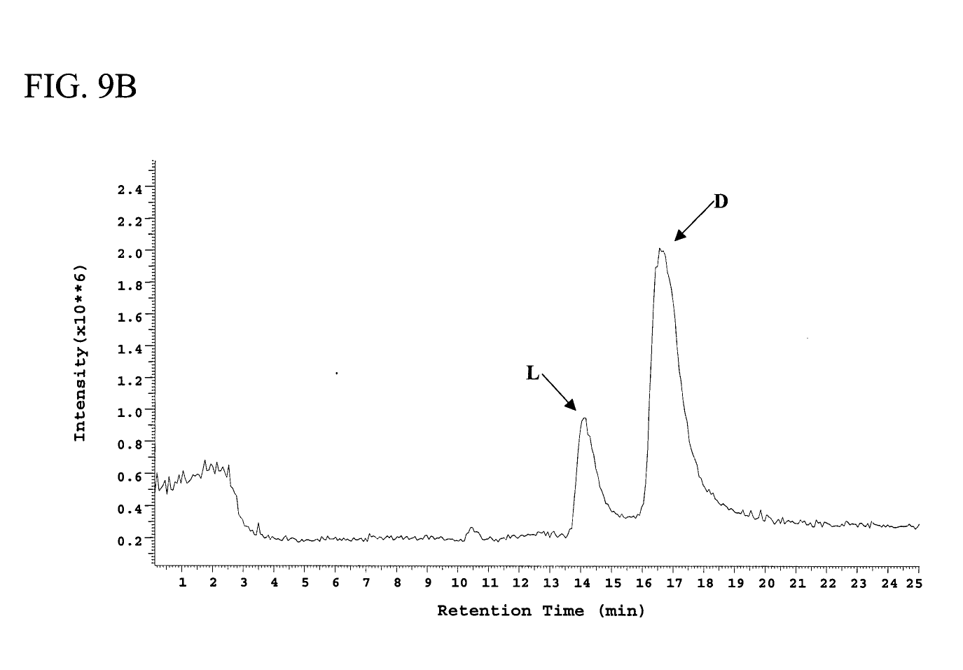

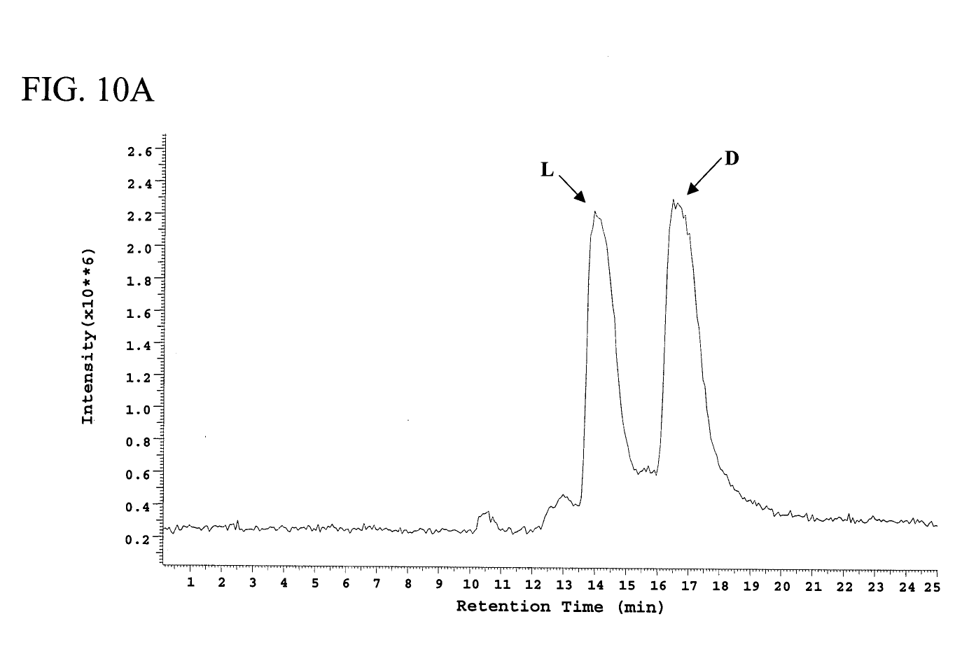

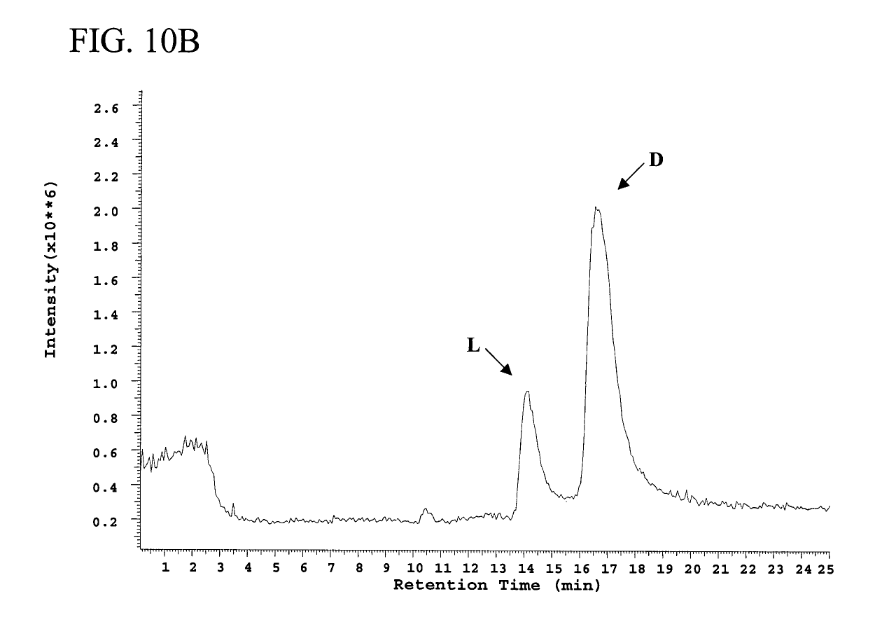

Concentrations of the esters of the enantiomers of the non-hydrolyzed HMB-propyl ester and HMB-butyl ester that resulted from the non-enzymatic and enzymatic hydrolysis reactions described above were also determined. FIGS. 9A and 9B illustrate the concentrations of the HMB-propyl ester enantiomers obtained after non-enzymatic and PPL catalyzed hydrolysis, respectively. Similarly, FIGS. 10A and 10B illustrate the concentrations of the HMB-butyl ester enantiomers obtained after non-enzymatic and PPL catalyzed hydrolysis, respectively. The chromatograms of FIGS. 9A and 10A illustrate both D- and L-HMB-ester enantiomers at equal concentrations, attesting to the non-enantioselective nature of chemical hydrolysis. By contrast, the chromatograms in FIGS. 9B and 10B illustrate the predominance of the D-ester enantiomer illustrating that the L-ester is enantioselectively hydrolyzed by PPL.

Conclusion: Lipase catalyzed hydrolysis of HMB-propyl ester and HMB-butyl ester is enantioselective. Thus, hydrolysis can be used for obtaining enantio-enrichment of HMB and other α-hydroxy acids such as the lactic acid under mild reaction conditions in aqueous systems.

### EXAMPLE 6

Enzyme Immobilization Technique and Results

Sodium alginate (500 mg), gelatin (300 mg) and water (9 ml) are introduced into and mixed in a 50 ml conical flask. The mix is then left undisturbed for 1 hour, after which the flask and its contents are autoclaved (120° C., 15 min.). The contents are then cooled at room temperature under continuous stirring. After the contents have cooled down, PPL (100 mg) in water (1 ml) is added to the flask and the contents stirred for further 15-20 min. Then glutaraldehyde (300 μl) is added to the flask contents and the mixture stirred for another 15 min. These contents are added drop-wise from a distance of around 10 cm by forcing them through the hypodermic needle in a 0.18M calcium chloride solution (2°-3° C.) . The gel breads solidify immediately upon contact with the calcium chloride solution, and after 30 min. are solidified. The solidified gel beads are washed 3-4 times with water to remove any loose enzyme from the surface.

### EXAMPLE 7

Synthesis of DL-HMB propyl ester

HMB acid (−10 grams) and anhydrous n-propyl alcohol (50 ml) were introduced into a three neck round bottom flask fitted with a condenser and heated to about 85° C. The reaction mixture was stirred by a magnetic needle and hydrogen chloride gas bubbled through the mixture for 30 minutes. (This process was done under completely anhydrous conditions). The mixture was then refluxed overnight. HMBPE was recovered by evaporating the alcohol on a rotary evaporator. Set forth in FIG. 11 is the RPC-8 chromatography of HMBPE produced in this example and used for further experiments as described in Example 8. The mobile phases chosen were water +0.1% trifluroacetic acid (TFA) and acetonitrile +0.1% TFA.

For analysis of enantio-enrichment of the HMB product, we analyzed the sample in a liquid chromatograph mass spectrometry (LCMS) system. Isocratic elution with a mobile phase composition of ammonium acetate buffer, methanol (70:30) and a chirobiotic TAG column was used for the total run time of 25 minutes.

Eventually a good yield of L-enriched HMB was observed.

### EXAMPLE 8

Hydrolysis of HMB Propyl Ester Using the Gel-Bound Enzyme Beads

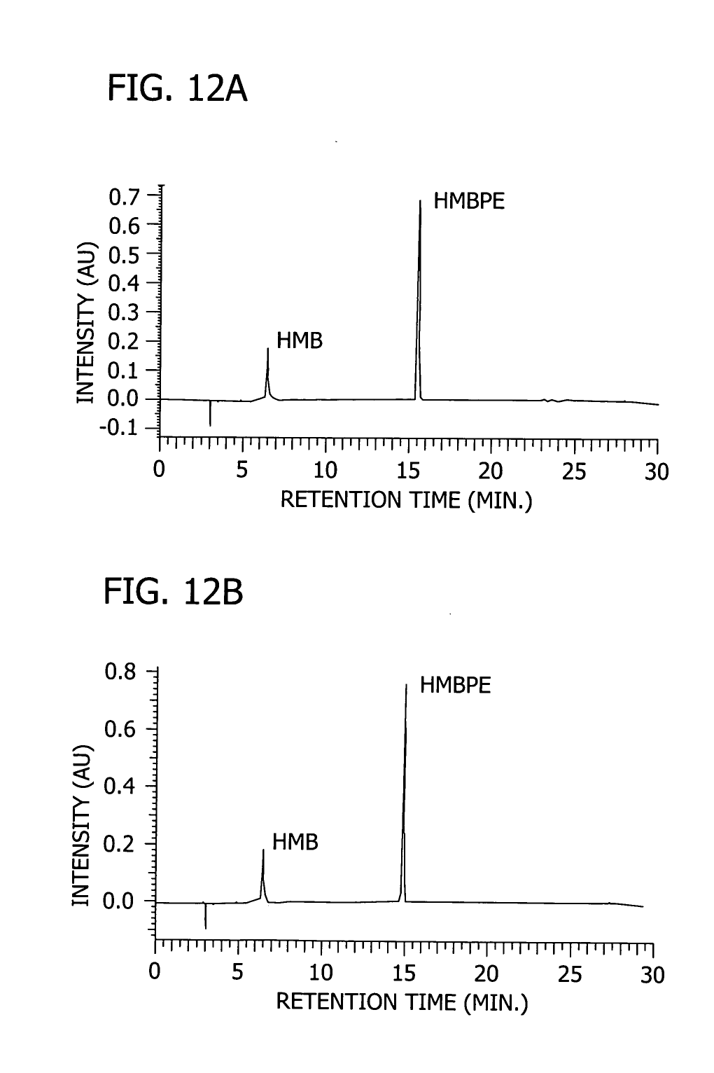

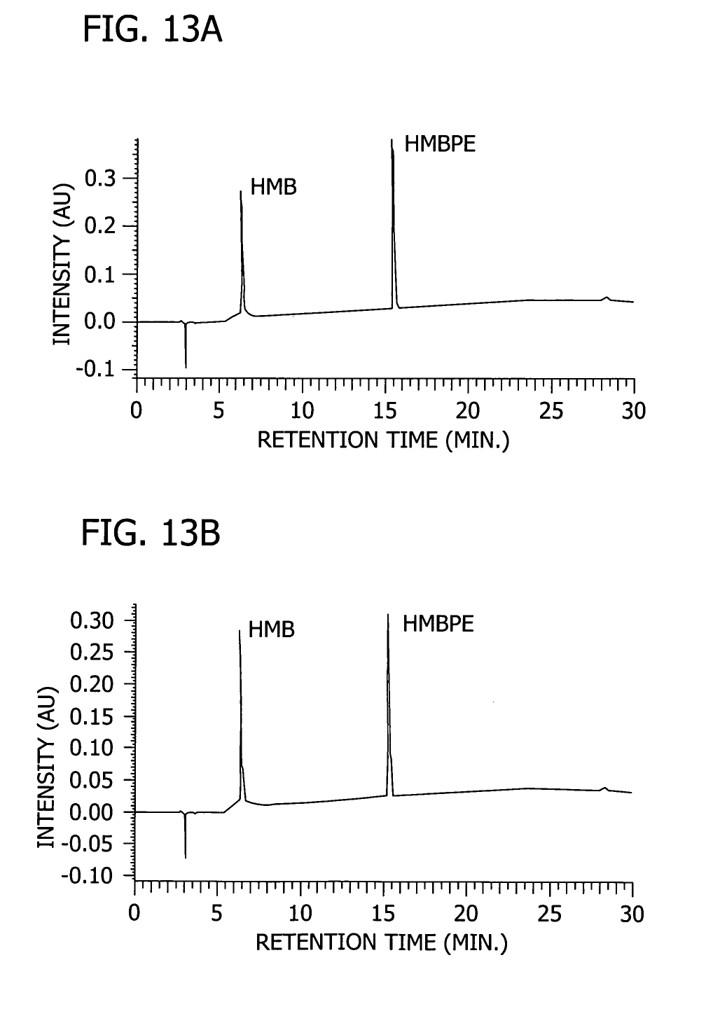

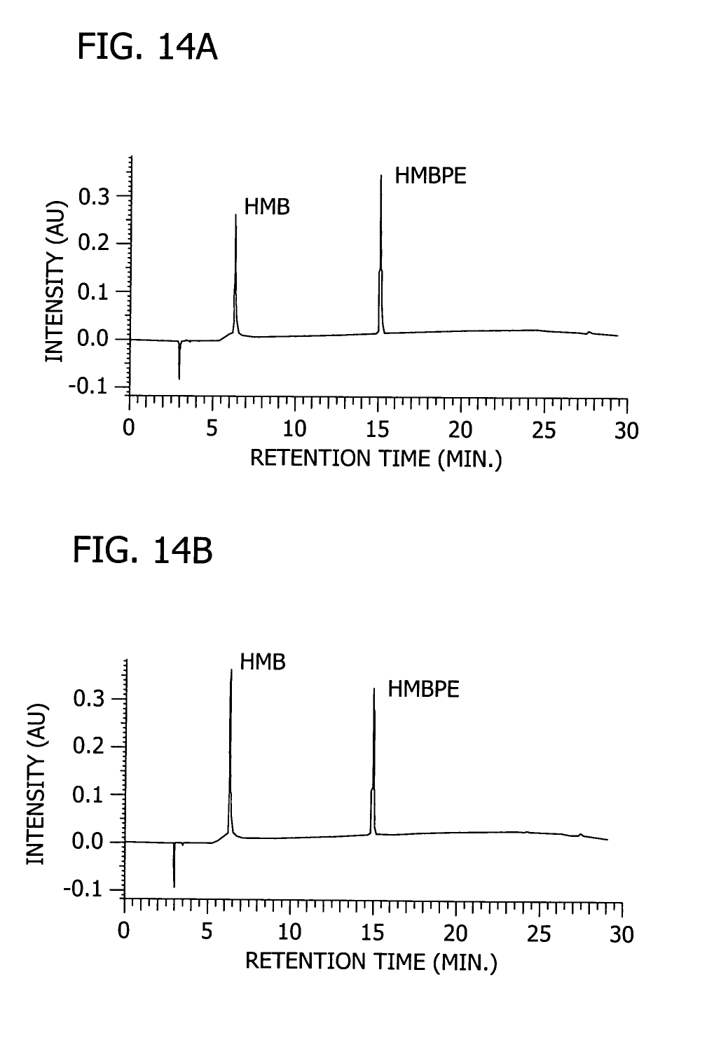

The immobilized enzyme catalyst beads as prepared in the manner described in Example 6 were placed in a conical flask. Water (20 ml) and HMB propyl ester (240 mg) were added to the flask and the contents were subjected to continuous shaking in a controlled temperature environment. This experiment was performed both in a controlled environment (incubated shaker at 37° C.), and at room temperature. Chromatographic analysis of the resulting products were compared with analyses of products obtained by hydrolysis with free enzyme (100 mg PPL) under the same time constraints. FIGS. 12A and 12B are the chromatographs of products obtained by contact of n-propyl HMB with free enzyme at room temperature after 2 hours and 3.30 hours respectively. FIGS. 13A and 13B are the chromatographs of products obtained by contact with alginate-immobilized beads (100 mg PPL) at room temperature after 2 hours and 3.30 hours respectively; and FIGS. 14A and 14B are the chromatographs of products obtained after 2 hours and 3.30 hours respectively in contact with the alginate immobilized enzyme (100 mg PPL) at 37° C.

The n-propyl ester product prior to enzymatic hydrolysis is a racemic mixture of 50% L form and 50% D form. So if the enzyme completely hydrolyzes the ester, it indicates that the reaction does not discriminate between the two different enantiomeric forms. Hydrolysis of HMBPE with PPL shows partial hydrolysis, indicating potential enantio-selectivity.

Reversed phase chromatography method was used for all the chromatograms set forth in the drawings. The mobile phases chosen were water +0.1% trifluroacetic acid (TFA) (solvent A) and acetonitrile +0.1% TFA (solvent B). The column was maintained at 30° C. The reaction was monitored at 220 nm and gradient elution for the mobile phases used was 0 to 55% solvent B in 30 min.

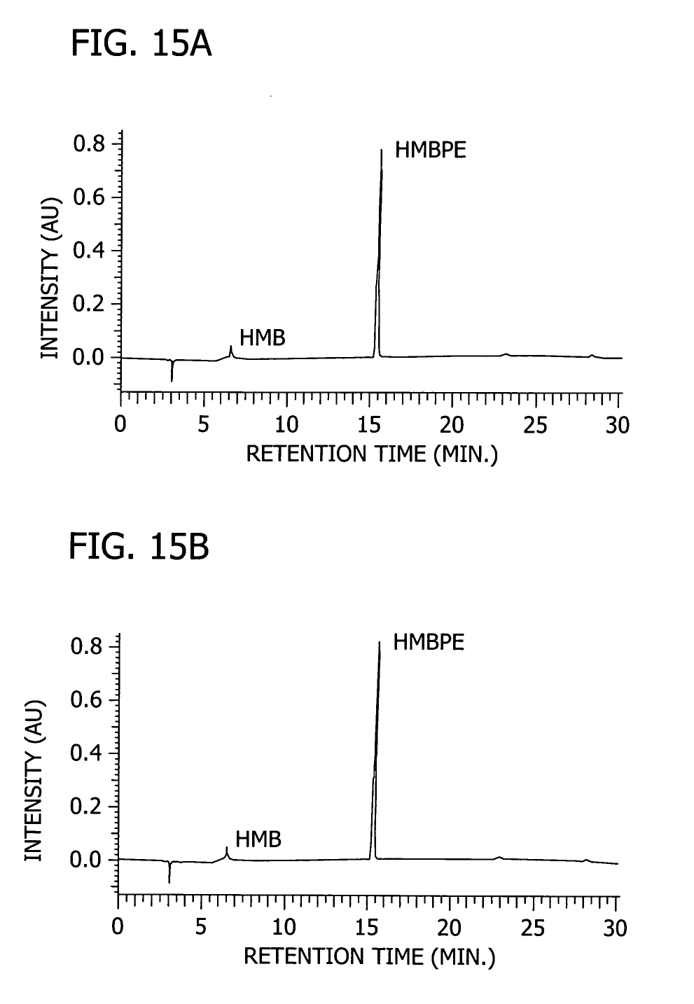

The hydrolysis of HMBP was also checked using alginate beads without enzymes(CONTROL). The gel beads were placed in a conical flask of water (20 ml) and HMB propyl ester (240 mg) were added to it. The mixture was thereafter maintained at a temperature of 37° C. The chromatograms of FIGS. 15A and 15B are obtained for the product formed, for the same time constraints as before.

As shown in FIGS. 15A and 15B, no hydrolysis of HMBP and HMB is found. HMB-propyl ester is thus a suitable substrate because it is not susceptible to chemical hydrolysis and has higher enzyme selectivity.

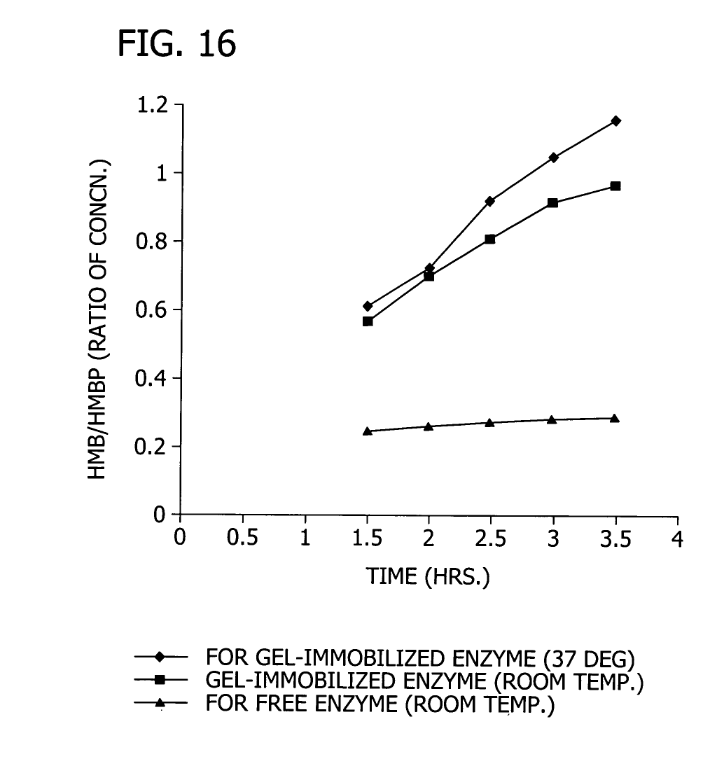

As compared to the earlier results in this field, enzyme in the immobilized form seems are capable of working better and faster than the free enzyme under typical hydrolysis conditions, as is evident from the chromatograms in FIGS. 15A and 15B. Since ester is a racemic mixture of 50% L form and 50% D form, a complete hydrolysis of the ester is undesirable, since it indicates that the reaction does not discriminate between the two different enantiomeric forms. Hydrolysis of HMBPE with immobilized PPL shows partial hydrolysis, indicating potential enantio-selectivity. As shown in FIGS. 16 and 15B, a 50% conversion of HMBPE to HMB acid is obtained after a run of 3 hrs. The reaction should be stopped after this since all HMB present should be L-enriched. Further hydrolysis beyond this point causes the D-enriched ester to be hydrolyzed, which is undesired.

Following is the comparative graph data for gel-immobilized (at 37° C.), gel-immobilized enzyme (at room temperature) and free enzyme hydrolysis of HMB propyl ester relative to the time fractions at which the sample products were collected.

Definitions

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Preferably, these moieties comprise 1 to 20 carbon atoms.

The “substituted hydrocarbyl” moieties described herein are hydrocarbyl moieties which are substituted with at least one atom other than carbon, including moieties in which a carbon chain atom is substituted with a hetero atom such as nitrogen, oxygen, silicon, phosphorus, boron, sulfur, or a halogen atom. These substituents include halogen, heterocyclo, alkoxy, alkenoxy, alkynoxy, aryloxy, hydroxy, protected hydroxy, keto, acyl, acyloxy; nitro, amino, amido, nitro, cyano, and thiol.

The alkyl groups described herein are preferably lower alkyl containing from one to six carbon atoms in the principal chain and up to 20 carbon atoms. They may be straight or branched chain and include methyl, ethyl, propyl, isopropyl, butyl, pentyl, hexyl, heptyl, and the like.

The alkenyl groups described herein are preferably lower alkenyl containing from two to six carbon atoms in the principal chain and up to 20 carbon atoms. They may be straight or branched chain and include ethenyl, propenyl, isopropenyl, butenyl, isobutenyl, hexenyl, and the like.

The alkynyl groups described herein are preferably lower alkynyl containing from two to six carbon atoms in the principal chain and up to 20 carbon atoms. They may be straight or branched chain and include ethynyl, propynyl, butynyl, isobutynyl, hexynyl, and the like.

The terms “aryl” or “ar” as used herein alone or as part of another group denote optionally substituted homocyclic aromatic groups, preferably monocyclic or bicyclic groups containing from 6 to 12 carbons in the ring portion, such as phenyl, biphenyl, naphthyl, substituted phenyl, substituted biphenyl or substituted naphthyl. Phenyl and substituted phenyl are the more preferred aryl.

The terms “halogen” or “halo” as used herein alone or as part of another group refer to chlorine, bromine, fluorine, and iodine.

The terms “heterocyclo” or “heterocyclic” as used herein alone or as part of another group denote optionally substituted, fully saturated or unsaturated, monocyclic or bicyclic, aromatic or nonaromatic hydrocarbon groups having at least one heteroatom in at least one ring, and preferably 5 or 6 atoms in each ring. The heterocyclo group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heterocyclo groups include furyl, thienyl, pyridyl and the like. Exemplary substituents include one or more of the following groups: hydrocarbyl, substituted hydrocarbyl, keto, hydroxy, protected hydroxy, acyl, acyloxy, alkoxy, alkenoxy, alkynoxy, aryloxy, halogen, amido, amino, nitro, cyano, and thiol.

The acyl moieties described herein contain hydrocarbyl, substituted hydrocarbyl or heterocyclo moieties.

The abbreviation “HMB” shall mean the 2-hydroxy analog of methionine, i.e., 2-hydroxy-4-(methylthio)butyric acid.

The terms “chiral,” “chiral configuration,” and “enantiomer” refer to a particular stereoisomer of a molecule. For example, L-methionine and L-HMB.

The term “identical chirality” or “identical chiral configuration” refers to the chiral carbon of two or more molecules having the same stereoisomeric configuration. For example, all L-isomers of α-amino acid have identical chiral configuration. Thus, in the general α-amino acid structure, R^{a}R^{b}C(NH₂)COOH, wherein R^{a} is hydrogen, hydrocarbyl, substituted hydrocarbyl or heterocyclo; and R^{b} is hydrogen, the —COOH, —NH2, R^{a}, and R^{b} constituents of L-isomers of α-amino acid have the same spatial arrangement around the chiral carbon. Similarly, the two or more L-enantiomers of a specific α-hydroxy carboxylic acid, such as two molecules of L-HMB, will have identical configuration to each other.

The term “enantiomeric excess” refers to a mixture comprising an D- and L-stereoisomers of an α-hydroxy acid or ester of an α-hydroxy acid wherein the mixture comprises an greater concentration of one stereoisomer over the other stereoisomer. For example, a mixture comprising D- and L-HMB isomers would have an enantiomeric excess of L-HMB wherein 19% of the HMB isomers are present in the D-isomeric form and 81% of the HMB isomers are present in the L-isomeric form.

The term “enantioselective” refers to the selection of a specific enantiomer of an enantiomeric mixture and interactions with said enantiomer. For example, an enantioselective enzyme, such as porcine pancreatic lipase (PPL), in an aqueous media selectively hydrolyzes L-HMB butyl esters to form HMB and butanol.

The term “PPL” refers to porcine pancreatic lipase.

1. A process for resolving an enantiomeric mixture of an α-hydroxy acid having the following formula: 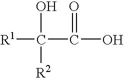
wherein R¹ is hydrogen, hydrocarbyl or substituted hydrocarbyl, wherein R² is hydrogen, hydrocarbyl or substituted hydrocarbyl, the process comprising:
contacting an aqueous feed composition comprising an enantiomeric mixture of esters of the α-hydroxy acid or derivatives thereof with an enantioselective enzyme selected from the group consisting of porcine pancreatic lipase, lipase from Aspergillus niger, lipase from Candida antarctica, and mixtures thereof which preferentially catalyzes hydrolysis of a first enantiomeric ester to produce a first α-hydroxy acid enantiomer corresponding to said first enantiomeric ester, thereby producing a reaction product comprising (i) the first α-hydroxy acid enantiomer and (ii) unreacted α-hydroxy acid ester, and
separating first α-hydroxy acid enantiomer from unreacted α-hydroxy acid ester.

2. A process as set forth in claim 1 wherein said aqueous feed composition is contacted with a heterogeneous catalyst comprising said enantioselective enzyme immobilized on a solid support.

3. A process as set forth in claim 2 wherein said aqueous feed composition is continuously or intermittently introduced into a catalytic reaction zone containing said heterogeneous catalyst, and said reaction product is continuously or intermittently withdrawn from said reaction zone.

4. A process as set forth in claim 3 wherein said heterogeneous catalyst comprises a plurality of catalyst bodies, each of said plurality of catalyst bodies comprising a porous support structure having said enzyme bound thereto or contained therewithin.

5. A process as set forth in claim 4 wherein said porous support structure comprises a composition selected from the group consisting of a polysaccharide, a polysaccharide/protein complex, a polysaccharide/protein complex cross-linked with a dialdehyde, and an alginate/gelatin complex.

6-13. canceled.

14. A process as set forth in claim 3 wherein said feed composition is continuously or intermittently introduced into a catalyst bed within said reaction zone, said catalyst bed comprising a heterogeneous catalyst comprising catalyst bodies on or within which said enzyme is immobilized; and a reaction mixture thus formed in the catalyst bed is caused to flow through the bed, said reaction product mixture being continuously or intermittently withdrawn from the bed.

15-16. canceled.

17. A process as set forth in claim 3 wherein said reaction product mixture withdrawn from the catalytic reaction zone is contacted in a phase extraction zone with an aqueous extractant and an organic extractant, thereby producing an aqueous extract and an organic extract, said first α-hydroxy acid enantiomer corresponding to said first enantiomeric ester being predominantly partitioned to said aqueous extract and said unreacted α-hydroxy acid ester being predominantly partitioned to said organic extract; and said organic extract is separated from said aqueous extract.

18. A process as set forth in claim 17 wherein said unreacted ester obtained in said organic extract is racemized and recycled by being introduced into said catalytic reaction zone.

19-20. canceled.

21. A process for the preparation of a heterogeneous catalyst for use in an enantiomerically selective hydrolysis of an α-hydroxy carboxylic acid, the process comprising:
preparing a premixture comprising an enantiomerically selective hydrolytic enzyme, an alkali metal saccharide and a protein;
heating the premixture to form a gel;
contacting the gel with a divalent cation, thereby forming a catalyst body comprising a solid porous support having said enzyme contained within the pores of the support; and
contacting enantiomeric mixture of an α-hydroxy acid ester with said catalyst body, thereby preferentially hydrolyzing a first enantiomeric ester of said mixture of esters to produce a first α-hydroxy acid enantiomer corresponding to said first ester.

22. A process as set forth in claim 21 wherein contacting of said gel with said divalent cation comprises:
forming drops of the gel; and
introducing the drops of gel into an aqueous bead formation medium comprising a divalent metal ion.

23. A process for resolving an enantiomeric mixture of an α-hydroxy acid having the following formula: 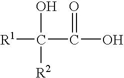
wherein R¹ is hydrogen, hydrocarbyl or substituted hydrocarbyl, wherein R² is hydrogen, hydrocarbyl or substituted hydrocarbyl, and wherein when R¹ is a substituted hydrocarbyl comprising a phosphorus atom, R² is hydrocarbyl or substituted hydrocarbyl, the process comprising:
forming a reaction mixture comprising (i) an enantioselective enzyme selected from the group consisting of porcine pancreatic lipase, lipase from Aspergillus niger, lipase from Candida antarctica, and mixtures thereof, and (ii) an enantiomeric mixture of esters of the α-hydroxy acid or derivatives thereof, wherein the enantioselective enzyme preferentially hydrolyzes a first enantiomeric ester to produce a first α-hydroxy acid enantiomer corresponding to said first enantiomeric ester,
forming a reaction product from the reaction mixture, the reaction product comprising (i) the first α-hydroxy acid enantiomer and (ii) unreacted α-hydroxy acid ester, and
separating the first α-hydroxy acid enantiomer and unreacted α-hydroxy acid ester from each other.

24. The process of claim 23, further comprising recovering the first α-hydroxy acid enantiomer. wherein the first α-hydroxy acid enantiomer is the L-isomer of the α-hydroxy acid.

25. (Canceled)

26. The process of claim 23, further comprising recovering the unreacted α-hydroxy acid ester and hydrolyzing the unreacted α-hydroxy acid ester to form an enantiomeric mixture having an enantiomeric excess of the D-isomer of the α-hydroxy acid.

27-33. canceled.

34. The process of claim 23, wherein the α-hydroxy analogs are α-hydroxy analogs of α-amino acids selected from the group consisting of methionine and lysine.

35. (Canceled)

36. The process of claim 23, wherein the first α-hydroxy acid enantiomer and the unreacted α-hydroxy acid ester or derivatives thereof are separated by subjection to a method selected from the group consisting of enantioselective chromatography, phase extraction, and two-phase extraction comprising an organic phase and an aqueous phase.

37-40. canceled.

41. The process of claim 23, wherein the pH of the reaction mixture is above at least about 5.

42-44. canceled.

45. The process of claim 23, wherein the temperature of the reaction mixture is above at least about 15° C.

46-49. canceled.

50. The process of claim 23, wherein the enantiomeric mixture of esters of the α-hydroxy acid is formed in an [a] esterification reaction mixture comprising the α-hydroxy acid and an alcohol.

51-55. canceled.

56. A stereoisomeric mixture having an enantiomeric excess of a stereoisomer of an α-hydroxy acid having the following formula: 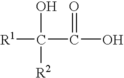
wherein R¹ is hydrogen, hydrocarbyl or substituted hydrocarbyl, wherein R² is hydrogen, hydrocarbyl or substituted hydrocarbyl, and wherein when R¹ is a substituted hydrocarbyl comprising a phosphorus atom, R² must be a hydrocarbyl or substituted hydrocarbyl, the stereoisomeric mixture being formed by a process comprising:
forming a reaction mixture comprising (i) an enantioselective enzyme selected from the group consisting of porcine pancreatic lipase, lipase from Aspergillus niger, lipase from Candida antarctica, and mixtures thereof, and (ii) an enantiomeric mixture of esters of the α-hydroxy acid, wherein the enantioselective enzyme preferentially hydrolyzes a first enantiomeric ester to produce a first mixture of α-hydroxy acid enantiomers that contains an enantiomeric excess of the first α-hydroxy acid enantiomer,
forming a reaction product from the reaction mixture comprising (i) the first mixture of α-hydroxy acid enantiomers, and (ii) unreacted second α-hydroxy acid ester,
separating the first mixture of α-hydroxy acid enantiomers and unreacted α-hydroxy acid ester enantiomers in the reaction product from each other, and
recovering the first mixture of α-hydroxy acid enantiomers to form the stereoisomeric mixture.

57. The stereoisomeric mixture of claim 56, wherein the first mixture of α-hydroxy acid enantiomers is comprised of an enantiomeric excess of the L-isomer of the α-hydroxy acid.

58. The stereoisomeric mixture of claim 56, further comprising recovering the unreacted α-hydroxy acid ester and hydrolyzing the unreacted α-hydroxy acid ester to form a second mixture of α-hydroxy acid enantiomers that has an enantiomeric excess of the D-isomer of the α-hydroxy acid.

59-65. canceled.

66. The stereoisomeric mixture of claim 56, wherein the α-hydroxy analog is selected from the group consisting of methionine and lysine.

67. The stereoisomeric mixture of claim 56, wherein the α-hydroxy analog comprises 2-hydroxy-4-(methylthio)butyric acid.

68. The stereoisomeric mixture of claim 56, wherein the hydrolyzed α-hydroxy acid or derivative thereof and the unreacted α-hydroxy acid ester or derivative thereof are separated by subjection to a method selected from the group consisting of enantioselective chromatography, phase extraction, and two-phase extraction comprising an organic phase and an aqueous phase.

69-72. canceled.

73. The stereoisomeric mixture of claim 56, wherein the pH of the reaction mixture is above at least about 5.

74-76. canceled.

77. The stereoisomeric mixture of claim 56, wherein the temperature of the reaction mixture is above at least about 15° C.

78-81. canceled.

82. The stereoisomeric mixture of claim 56 wherein the enantiomeric mixture of esters of the α-hydroxy acid or derivative thereof is formed in a esterification reaction mixture comprising the α-hydroxy acid or derivative thereof and an alcohol.

83-87. canceled.

88. The stereoisomeric mixture of claim 56 wherein at least 60% of the first mixture α-hydroxy acid enantiomers are the L-isomer of α-hydroxy acid.

89-91. canceled.

92. The stereoisomeric mixture of claim 56 wherein at least 60% of the second mixture α-hydroxy acid enantiomers are the D-isomer of α-hydroxy acid.

93-95. canceled.

96. A feed supplement comprising the stereoisomeric mixture of claim 56.

97. The feed supplement of claim 96 wherein at least 60% of the first mixture of α-hydroxy acid enantiomers are the L-isomer of α-hydroxy acid.

98-100 canceled.

101. A feed supplement comprising the second mixture of α-hydroxy acid enantiomers of claim 56 wherein the first mixture of α-hydroxy acid enantiomers has an enantiomeric excess of the D-isomer of the α-hydroxy acid.

102. The feed supplement of claim 101 wherein at least 60% of the second mixture α-hydroxy acid enantiomers are the D-isomer of α-hydroxy-acid.

103-105 canceled.

106. A process for supplementing the diet of an animal, the process comprising providing the feed supplement of claim 96 to the animal.

107-112 canceled.

113. A process for producing and resolving an α-hydroxy acid enantiomer or derivative thereof in an enantiomeric mixture, the process comprising:
forming a first reaction mixture comprising an α-hydroxy acid and an alcohol,
forming a first product mixture from the first reaction mixture, the first product mixture comprising an α-hydroxy ester corresponding to the α-hydroxy acid,
forming a second reaction mixture from the first product mixture, the second reaction mixture comprising the α-hydroxy ester and an enantioselective enzyme,
forming a second product mixture from the second reaction mixture, the second product mixture comprising a first α-hydroxy acid and unhydrolyzed α-hydroxy ester, wherein the first α-hydroxy acid is produced by the enantioselective hydrolysis of a first enantiomer of the α-hydroxy ester, and
separating the first α-hydroxy acid and unhydrolyzed α-hydroxy ester in the second product mixture from each other.

114. The process of claim 113 wherein the α-hydroxy acid in the first reaction mixture is selected from the group consisting of glycolic acid, lactic acid, glyceric acid, tartaric acid, citric acid, glyoxylic acid, pyruvic acid, mandelic acid, malic acid, benzilic acid; α-hydroxy analogs of asparagine, glycine, alanine, valine, leucine, isoleucine, phenylalanine, proline, serine, threonine, cysteine, methionine, tryptophan, tyrosine, glutamine, aspartic acid, glutamic acid, lysine, arginine, and histidine; and derivatives thereof.

115-119. canceled.

120. The process of claim 113 wherein the enantioselective enzyme is a lipase enzyme.

121-125. canceled.

26. The process of claim 113 wherein the first reaction mixture is at least about 65° C.

127-129. canceled.

130. The process of claim 113 wherein the second reaction mixture has a pH of at least about 5.

131-133. canceled.

134. The process of claim 113 wherein the second reaction mixture is at least about 15° C.

135-144 canceled.

145. The process of claim 113 wherein the enantioselective enzyme is immobilized and the second reaction mixture comprising the α-hydroxy ester is continuously contacted with the enantioselective enzyme to continuously hydrolyze the α-hydroxy acid ester to produce the second product mixture comprising the first α-hydroxy acid and unhydrolyzed α-hydroxy ester.

146. The process of claim 145 wherein the first α-hydroxy acid is continuously removed from the second product mixture.

147. A process for producing and continuously resolving an α-hydroxy acid enantiomer or derivative thereof in an enantiomeric mixture, the process comprising:
forming a first reaction mixture comprising an α-hydroxy acid and an alcohol,
forming a first product mixture from the first reaction mixture, the first product mixture comprising an α-hydroxy ester corresponding to the α-hydroxy acid,
forming a second reaction mixture from the first product mixture, the second reaction mixture comprising the α-hydroxy ester,
forming a second product mixture by continuously contacting the second reaction mixture with an immobilized enantioselective enzyme, the second product mixture comprising a first α-hydroxy acid and unhydrolyzed α-hydroxy ester, wherein the first α-hydroxy acid is produced by the enantioselective hydrolysis of a first enantiomer of the α-hydroxy ester, and continuously separating the α-hydroxy acid produced by the enantioselective hydrolysis of the α-hydroxy ester from the unreacted α-hydroxy ester.

148-149. canceled.

150. The process of claim 147 wherein the first α-hydroxy acid and unreacted α-hydroxy ester are intermittently removed from the reaction zone.

151. The process of claim 147 wherein the first α-hydroxy acid and unreacted α-hydroxy ester are continuously removed from the reaction zone.

152. The process of claim 147 wherein the second reaction mixture comprising the α-hydroxy ester is continuously introduced to the immobilized enantioselective enzyme within a reaction zone in which the α-hydroxy ester is hydrolyzed to produce the first α-hydroxy acid.

153-154. canceled.

155. A process for resolving an enantiomeric mixture of an α-hydroxy acid having the following formula: 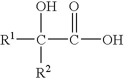
wherein R¹ is hydrogen, hydrocarbyl or substituted hydrocarbyl, and wherein R² is hydrogen, hydrocarbyl or substituted hydrocarbyl, the process comprising:
forming a reaction mixture comprising (i) a heterogeneous catalyst comprising the enantioselective enzyme immobilized on a solid support, and (ii) an enantiomeric mixture of esters of the α-hydroxy acid or derivatives thereof, wherein the enantioselective enzyme preferentially hydrolyzes a first enantiomeric ester to produce a first α-hydroxy acid enantiomer corresponding to said first enantiomeric ester,
forming a reaction product from the reaction mixture, the reaction product comprising (i) the first α-hydroxy acid enantiomer and (ii) unreacted α-hydroxy acid ester, and
separating the first α-hydroxy acid enantiomer and unreacted α-hydroxy acid ester from each other.

156. The process of claim 155 wherein the enantiomeric mixture is continuously or intermittently introduced into a catalytic reaction zone containing said heterogeneous catalyst and wherein the reaction product is continuously or intermittently withdrawn from said reaction zone.

157. The process of claim 155 wherein said heterogeneous catalyst comprises a plurality of catalyst bodies, each of said plurality of catalyst bodies comprising a porous support structure having said enzyme bound thereto or contained therewithin.

158. A process for resolving an enantiomeric mixture of an c-hydroxy acid, wherein the α-hydroxy acid is selected from the group consisting of glycolic acid, lactic acid, glyceric acid, tartaric acid, citric acid, glyoxylic acid, pyruvic acid, malic acid, benzilic acid; α-hydroxy analogs of asparagine, alanine, valine, isoleucine, phenylalanine, proline, serine, threonine, cysteine, methionine, tryptophan, tyrosine, glutamine, aspartic acid, glutamic acid, lysine, arginine, and histidine; and derivatives thereof, the process comprising:
contacting an aqueous feed composition comprising an enantiomeric mixture of esters of the α-hydroxy acid or derivatives thereof with an enantioselective enzyme which preferentially catalyzes hydrolysis of a first enantiomeric ester to produce a first α-hydroxy acid enantiomer corresponding to said first enantiomeric ester, thereby producing a reaction product comprising (i) the first α-hydroxy acid enantiomer and (ii) unreacted α-hydroxy acid ester, and
separating first α-hydroxy acid enantiomer from unreacted α-hydroxy acid ester.

159. A process as set forth in claim 158 wherein said aqueous feed composition is contacted with a heterogeneous catalyst comprising said enantioselective enzyme immobilized on a solid support.

160. A process as set forth in claim 159 wherein said aqueous feed composition is continuously or intermittently introduced into a catalytic reaction zone containing said heterogeneous catalyst, and wherein said reaction product is continuously or intermittently withdrawn from said reaction zone.

161. A process as set forth in claim 159 wherein said heterogeneous catalyst comprises a plurality of catalyst bodies, each of said plurality of catalyst bodies comprising a porous support structure having said enzyme bound thereto or contained therewithin.

162. The process of claim 161 wherein said porous support structure comprises a composition selected from the group consisting of a polysaccharide, a polysaccharide,/protein complex, a polysaccharide/protein complex cross-linked with a dialdehyde, and an alginate/gelatin complex.

163. A process as set forth in claim 160 wherein said feed composition is continuously or intermittently introduced into a catalyst bed within said reaction zone, said catalyst bed comprising a heterogeneous catalyst comprising catalyst bodies on or within which said enzyme is immobilized; and a reaction mixture thus formed in the catalyst bed is caused to flow through the bed, said reaction product mixture being continuously or intermittently withdrawn from the bed.

164. A process as set forth in claim 160 wherein said reaction product mixture withdrawn from the catalytic reaction zone is contacted in a phase extraction zone with an aqueous extractant and an organic extractant, thereby producing an aqueous extract and an organic extract, said first α-hydroxy acid enantiomer corresponding to said first enantiomeric ester being predominantly partitioned to said aqueous extract and said unreacted α-hydroxy acid ester being predominantly partitioned to said organic extract; and said organic extract is separated from said aqueous extract.

165. A process as set forth in claim 164 wherein said unreacted ester obtained in said organic extract is racemized and recycled by being introduced into said catalytic reaction zone.

166. The process of claim 158, further comprising recovering the first α-hydroxy acid enantiomer, wherein the first α-hydroxy acid enantiomer is the L-isomer of the α-hydroxy acid.

167. The process of claim 158, further comprising recovering the unreacted α-hydroxy acid ester and hydrolyzing the unreacted α-hydroxy acid ester to form an enantiomeric mixture having an enantiomeric excess of the D-isomer of the α-hydroxy acid.

168. The process of claim 158, wherein the enantioselective enzyme is a lipase enzyme.

169. The process of claim 158, wherein the α-hydroxy analogs are α-hydroxy analogs of α-amino acids selected from the group consisting of methionine and lysine.

170. The process of claim 158, wherein the first α-hydroxy acid enantiomer and the unreacted α-hydroxy acid ester or derivatives thereof are separated by subjection to a method selected from the group consisting of enantioselective chromatography, phase extraction, and two-phase extraction comprising an organic phase and an aqueous phase.

171. The process of claim 158, wherein the pH of the reaction mixture is above at least about 5.

172. The process of claim 158, wherein the temperature of the reaction mixture is above at least about 15° C.

173. The process of claim 158, wherein the enantiomeric mixture of esters of the α-hydroxy acid is formed in an esterification reaction mixture comprising the α-hydroxy acid and an alcohol.

174. A stereoisomeric mixture having an enantiomeric excess of a stereoisomer of an α-hydroxy acid, wherein the α-hydroxy acid is selected from the group consisting of glycolic acid, lactic acid, glyceric acid, tartaric acid, citric acid, glyoxylic acid, pyruvic acid, malic acid, benzilic acid; α-hydroxy analogs of asparagine, alanine, valine, isoleucine, phenylalanine, proline, serine, threonine, cysteine, methionine, tryptophan, tyrosine, glutamine, aspartic acid, glutamic acid, lysine, arginine, and histidine; and derivatives thereof, the stereoisomeric mixture being formed by a process comprising:
forming a reaction mixture comprising (i) an enantioselective enzyme, and (ii) an enantiomeric mixture of esters of the α-hydroxy acid, wherein the enantioselective enzyme preferentially hydrolyzes a first enantiomeric ester to produce a first mixture of α-hydroxy acid enantiomers that contains an enantiomeric excess of the first α-hydroxy acid enantiomer,
forming a reaction product from the reaction mixture comprising (i) the first mixture of α-hydroxy acid enantiomers, and (ii) unreacted second α-hydroxy acid ester,
separating the first mixture of (-hydroxy acid enantiomers and unreacted α-hydroxy acid ester enantiomers in the reaction product from each other, and
recovering the first mixture of α-hydroxy acid enantiomers to form the stereoisomeric mixture.

175. The stereoisomeric mixture of claim 174, wherein the first mixture of α-hydroxy acid enantiomers is comprised of an enantiomeric excess of the L-isomer of the α-hydroxy acid.

176. The stereoisomeric mixture of claim 174, further comprising recovering the unreacted α-hydroxy acid ester and hydrolyzing the unreacted α-hydroxy acid ester to form a second mixture of α-hydroxy acid enantiomers that has an enantiomeric excess of the D-isomer of the α-hydroxy acid.

177. The stereoisomeric mixture of claim 174, wherein the enantioselective enzyme is a lipase enzyme.

178. The stereoisomeric mixture of claim 174, wherein the α-hydroxy analog is selected from the group consisting of methionine and lysine.

179. The stereoisomeric mixture of claim 174, wherein the α-hydroxy analog comprises 2-hydroxy-4-(methylthio)butyric acid.

180. The stereoisomeric mixture of claim 174, wherein the hydrolyzed α-hydroxy acid or derivative thereof and the unreacted α-hydroxy acid ester or derivative thereof are separated by subjection to a method selected from the group consisting of enantioselective chromatography, phase extraction, and two-phase extraction comprising an organic phase and an aqueous phase.

181. The stereoisomeric mixture of claim 174, wherein the pH of the reaction mixture is above at least about 5.

182. The stereoisomeric mixture of claim 174, wherein the temperature of the reaction mixture is above at least about 15° C.

183. The stereoisomeric mixture of claim 174, wherein the enantiomeric mixture of esters of the α-hydroxy acid or derivative thereof is formed in a esterification reaction mixture comprising the α-hydroxy acid or derivative thereof and an alcohol.

184. The stereoisomeric mixture of claim 174 wherein at least 60% of the first mixture α-hydroxy acid enantiomers are the L-isomer of α-hydroxy acid.

185. The stereoisomeric mixture of claim 174 wherein at least 60% of. the second mixture α-hydroxy acid enantiomers are the D-isomer of α-hydroxy acid.

186. A feed supplement comprising the stereoisomeric mixture of claim 174.

187. The feed supplement of claim 186 wherein at least 60% of the first mixture of α-hydroxy acid enantiomers are the L-isomer of α-hydroxy acid.
